(19) United States
(12) Patent Application Publication
(10) Pub. No.: US 2025/0325535 A1
(43) Pub. Date: Oct. 23, 2025

(54) IDENTIFICATION OF NOVEL SMALL-MOLECULE INHIBITORS OF SARS-CoV-2 BY CHEMICAL GENETICS

(71) Applicants: The University of Hong Kong, Hong Kong (HK); Centre for Virology, Vaccinology and Therapeutics Limited, Hong Kong (HK)

(72) Inventors: Shuofeng Yuan, Hong Kong (HK); Jasper Fuk-Woo Chan, Hong Kong (HK); Kwok Yung Yuen, Hong Kong (HK)

(21) Appl. No.: 19/187,186

(22) Filed: Apr. 23, 2025

Related U.S. Application Data
(60) Provisional application No. 63/637,387, filed on Apr. 23, 2024.

(51) Int. Cl. A61K 31/473; A61K 31/403; A61P 31/14

(52) U.S. Cl. CPC A61K 31/473; A61K 31/403; A61P 31/14

(57) ABSTRACT

This invention provides a novel small-molecule allosteric protease inhibitor, 10-(4-methylphenyl)-7-phenyl-6,7,8,10-tetrahydro-5H-indeno[1,2-b]quinoline-9,11-dione, which exhibits the highest selectivity index (SI), and shows inhibition against several SARS-CoV-2 variants of concern (VOC) and multiple human coronaviruses including MERS-CoV, SARS-CoV, and HCoV-229E. Not only does it demonstrate antiviral potency against a wide spectrum of coronavirus strains and species, but it also shows drug synergism with nirmatrelvir, which could be used in combination therapy to not only treat coronavirus-caused infections and diseases, but also lower the risk of antiviral resistance.

### CROSS REFERENCE TO RELATED APPLICATIONS

This application claims benefit of U.S. Provisional Application No. 63/637,387 filed Apr. 23, 2024, which is hereby incorporated herein by reference in its entirety.

### REFERENCE TO SEQUENCE LISTING

The Sequence Listing XML submitted as a file named “UHK_01516US_ST26.xml,” created on Apr. 10, 2025, and having a size of 11,733 bytes is hereby incorporated by reference pursuant to 37 C.F.R. § 1.834(c)(1).

### FIELD OF THE INVENTION

The present invention relates to coronavirus-targeting antiviral molecules. More specifically, one allosteric protease inhibitor compound is identified as a novel small-molecule inhibitor, exhibiting pan-coronavirus antiviral properties and potential in use with nirmatrelvir.

### BACKGROUND

In the past 20 years, the human society has encountered three significant spillovers of novel coronaviruses: SARS-CoV, MERS-CoV, and SARS-CoV-2 (7-9). Among these outbreaks, the COVID-19 pandemic caused by SARS-CoV-2 has resulted in millions of cases and fatalities, and caused a severe impact on the global economy. At the dawn of COVID-19 outbreak, patients typically exhibited symptoms such as cough, congestion, fatigue, fever, breathing difficulties, ground-glass opacity found in lung CT scans (10-13). Some patients also experienced diarrhoea, confusion, abnormal liver, and renal functions (10, 12, 13). However, as the pandemic evolved, COVID-19 has gradually become an upper respiratory tract infection in most cases and symptoms are often mild (14).

Although the world is moving towards a post-COVID phase, it is crucial to be prepared for the emergence of a new variant of SARS-CoV-2 or another zoonotic spillover of coronaviruses from wild reservoirs. One of the crucial measures to control emerging coronavirus outbreaks is to develop effective antivirals with broad-spectrum activity.

Currently, there are only three major druggable protein targets in SARS-CoV-2: Spike protein, 3CL-protease (3CLpro) and RNA-dependent RNA polymerase (RdRp) (15-17). Most monoclonal antibodies initially developed for the treatment of COVID-19 by targeting the Spike protein have failed, due to the emergence of the Omicron variant (18).

There are eight approved small molecule antiviral drugs, yet they are either conventional RdRp or 3CLpro inhibitors (16, 19-21). Four of them (Remdesivir, JT001, Molnupiravir and Azvudine) are nucleotide analogues (22-25), which could be detrimental to the dividing cells (5, 6). Four of them are 3CLpro inhibitors (Nirmatrelvir-ritonavir, Ensitrelvir, Leritrelvir and Simnotrelvir-Ritonavir) (16, 26-28). Nirmatrelvir-ritonavir (Paxlovid) is a covalent protease inhibitor, while Ensitrelvir is a non-covalent inhibitor that binds to the substrate-binding pocket of 3CLpro (16, 26). Despite the potent antiviral activity of these drugs, none of them are designed for SARS-CoV-2 specifically and resistance have been reported in some in vitro studies (2, 3). Therefore, it is essential to continue the search for novel antivirals.

It is an object of the invention to provide compositions and methods for the treatment of SARS-CoVs, particularly SARS-CoV-2.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

Compositions and methods of treating a subject for a coronavirus infection are provided.

The compositions include an effective amount of a small-molecular allosteric protease inhibitor, 10-(4-methylphenyl)-7-phenyl-6,7,8,10-tetrahydro-5H-indeno[1,2-b]quinoline-9,11-dione (compound 172, having formula (I) as below), or a functionally equivalent derivative thereof, which exhibited the highest selectivity index (SI), and showed inhibition against several SARS-CoV-2 variants of concern (VOC) and multiple human coronaviruses including MERS-CoV, SARS-CoV, and HCoV-229E, when compared to other compounds tested.

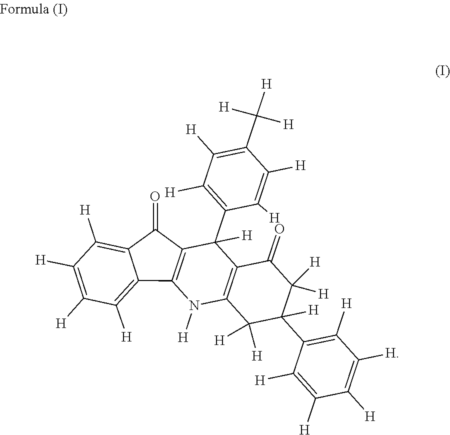

In some forms, the composition further comprises nirmatrelvir, which displays drug synergism with compound 172.

A method of treating coronavirus infections and/or preventing growth of coronavirus is further provided herewith, comprising administering a composition which comprises compound 172 having Formula (I) above.

The amount of compound 172 or derivative thereof, or a pharmaceutically acceptable salt thereof can be effective to, for example, reduce viral replication, reduce one or more symptoms of a disease, disorder, or illness associated with virus, or a combination thereof. Symptoms include, but are not limited to, fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhoea, tiredness, nausea, vomiting, and combinations thereof. The subject can be, for example, a mammal or a bird. In preferred embodiments, the subject is a human.

The subject can be symptomatic or asymptomatic. In some embodiments, the subject has been, or will be, exposed to the virus. In some embodiments, treatment begins 1, 2, 3, 4, 5, or more hours, days, or weeks prior to or after exposure to the virus. In some embodiments, the subject has not been exposed to the virus. In some embodiments, the subject anticipates being exposed to the virus. Thus, preventative and prophylactic methods are also provided, and are included in the term ‘treatment’.

In a further embodiment, the coronavirus includes SARS-CoV-2 VOC Delta, Omicron BA.1/BA.5, MERS-CoV, SARS-CoV and HCoV-299E.

The virus can be a Severe acute respiratory syndrome-related coronavirus, a Bat Hp-betacoronavirus Zhejiang2013, a Rousettus bat coronavirus GCCDC1, a Rousettus bat coronavirus HKU9, Eidolon bat coronavirus C704, a Pipistrellus bat coronavirus HKU5, a Tylonycteris bar coronavirus HKU4, a Middle East respiratory syndrome-related coronavirus, a Hedgehog coronavirus, a murine coronavirus, a Human coronavirus HKU1, a China Rattus coronavirus HKU24, a Betacoronavirus 1, a Myodes coronavirus 2JL14, a Human coronavirus NL63, a Human coronavirus 229E, or a Human coronavirus OC43.

In preferred embodiments, the virus is a Severe acute respiratory syndrome-related coronavirus, such as SARS-CoV-2, SARS-CoV, SARSr-CoV RaTG13, SARS-CoV PC₄₋₂₂7, or SARSr-CoV BtKY72.

In some embodiments, the virus is a SARS-CoV-2 having a genome encoded by a nucleic acid sequence having at least 85%, 86%, 87%, 88%, 89%, 90%, 91%, 92%, 93%, 94%, 95%, 96%, 97%, 98%, 99%, or 100% sequence identity to the DNA sequence of GenBank Accession No. MN908947.3 or GenBank Accession No. MN985325.1.

In some embodiments, the Severe acute respiratory syndrome-related coronavirus is SARS-CoV, for example, a SAR-CoV having a genome encoded by a nucleic acid sequence having at least 85%, 86%, 87%, 88%, 89%, 90%, 91%, 92%, 93%, 94%, 95%, 96%, 97%, 98%, 99%, or 100% sequence identity to the DNA sequence of GenBank Accession No. AY274119.3.

In some embodiments, the virus is a Middle East respiratory syndrome-related coronavirus, for example, a MERS-CoV having a genome encoded by a nucleic acid sequence having at least 85%, 86%, 87%, 88%, 89%, 90%, 91%, 92%, 93%, 94%, 95%, 96%, 97%, 98%, 99%, or 100% sequence identity to the DNA sequence of GenBank Accession No. JX869059.2.

In some embodiments, the subject has a disease or disorder associated with the virus. For example, in embodiments, a subject exposed or infected with SARS-CoV-2 has COVID-19. In some embodiments, the subject is not infected with influenza.

In some embodiments, a viral infection is detected or diagnosed in a subject prior to, during, or after treatment. Detection and diagnosis of viral infection may include, but is not limited to, PCR tests designed to detect viral RNA, and serological and immunodiagnostic tests designed to detect antibodies against the virus. An exemplary test for SARS-CoV-2 infection/COVID-19 is the iAMP COVID-19 Detection Kit, which is a real-time fluorescent isothermal assay for use on raw samples without RNA extraction.

In some embodiments, the subject has been in close contact with a person that has tested positive for the virus. Such a person may or may not be exhibiting one or more symptoms of an infection. In some embodiments, the subject of the treatment is identified by contact-tracing as having been exposed to the virus, or one or more persons infected therewith.

The compound 172 or derivative thereof, or pharmaceutically acceptable salt thereof is typically administered in a pharmaceutical composition including a pharmaceutically acceptable carrier and/or excipient. Thus, pharmaceutical compositions are also provided. Dosage forms are also provided and include, but not limited to tablets of compound 172 or derivative thereof, or pharmaceutically acceptable salt thereof. In some embodiments, the subject is administered a dose dosage form of compound 172 or derivative thereof, or pharmaceutically acceptable salt thereof 1, 2, 3, 4, or 5 times per day. In some embodiments, the dosage regimen is a pulse dosage regimen that includes 1, 2, 3, or more large bolus doses in close proximity (e.g., at most 5, 10, 15, 30, 45, or 60 minutes, or 1, 2, 3, 4, 5, 6, or more hours apart). In some embodiments, the bolus doses are followed by a drug administration holiday (e.g. at least 12 hours, or 1, 2, 3, 4, 5, or more days), optionally until the drug level in the subject's serum drops to zero or near zero (e.g., no more than 1%, 5%, 10%, or 20% of the peak serum level).

The compound 172 or derivative thereof, or a pharmaceutically acceptable salt thereof can be administered systemically or locally. Exemplary routes of administration include, but are not limited to, oral, parenteral, topical or mucosal. In some embodiments, the composition is administered to lungs (e.g., pulmonary administration) by oral inhalation or intranasal administration. In some embodiments, the composition is administered intranasally to the nasal mucosa.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The disclosed method and compositions can be understood more readily by reference to the following detailed description of particular embodiments and the Example included therein and to the Figures and their previous and following description.

FIG. 1A-1C. Phenotype-based high-throughput antiviral screening. FIG. 1A. Primary screening results using a CPE-inhibition assay. The primary screening cutoff is set as over 60% cell viability in both duplicates. Out of 50,213 compounds, 168 are identified to satisfy this criterion. Mock infection is included to normalize the cell viability (100%). Remdesivir is added at a concentration of 10 μM as positive control (shown in red). FIG. 1B. Plaque reduction assay results of the five finalized compounds. VeroE6 cells are inoculated with 40 PFU of SARS-CoV-2 D614G virus and subsequently treated with each of the five selected hit compounds, which are diluted from 20 μM to 1.25 μM in 2-fold intervals. At 72 hpi, the cells are fixed with 10% formalin/PBS and stained with a 0.5% crystal violet solution to visualize the plaques. The IC50 value is calculated by fitting the number of plaque formations against the logarithmic concentrations of the compounds using a non-linear regression model in GraphPad Prism software. FIG. 1C. Antiviral profiles of the five selected compounds with their chemical structure, IC₅₀ (by plaque reduction assay), CC₅₀ (by MTT cytotoxicity assay) and selectivity index (SI) as shown. The highest tested concentration in MTT cytotoxicity assay is 100 μM due to the maximal water solubility in 1% DMSO.

FIG. 2A-2E. Pan-coronavirus activity and raise of escape mutant of compound 172. FIG. 2A. viral load reduction assay conducted against SARS-CoV-2 VOC Delta, Omicron BA.1/BA.5, and co-plotted with cytotoxicity using MTT cytotoxicity assays. The viral yield is expressed as percentage of DMSO control. Compound 172 is diluted in 2-fold intervals from 20 μM to 0.3125 μM, and the IC50 value is calculated by fitting the normalized viral load against the logarithmic concentrations using a non-linear regression model in GraphPad Prism software. MTT cytotoxicity assays are carried out as previously described. FIG. 2B. VeroE6 or Huh7 cells are infected with MERS-CoV, SARS-CoV, or HCoV-229E at MOI 0.01. After 48 hpi, the supernatant and cell lysate are collected and lysed, and the viral RNA copies are measured using RT-qPCR. FIG. 2C. Antiviral activity of Nirmatrelvir as a reference control of compound 172. FIG. 2D. Vero-TMPRSS2 cells are infected either by Passage 0 (D614G virus) or Passage 6 virus at MOI 0.01, followed by treatment with varying concentrations of compound 172. After 48 hours, the supernatant is collected and the viral load is measured using RT-qPCR. Statistical analysis for all the assays above are performed by Student's T-test: *** P<0.001; **P<0.01; *P<0.05. FIG. 2E. Nanopore sequencing results. 84% virus population carried S301P point mutation in the 3CLpro. 172 Escape mutant is raised in six passages in Vero-TMPRSS2, by reducing MOI and increasing 172 concentrations in each passage, until the CPE inhibition effect of 172 had abolished.

FIG. 3A-3D. Characterization of compound 172-resistant S301P recombinant SARS-CoV-2. FIG. 3A. Plaque morphology of recombinant WT and 3CLpro S301P virus. VeroE6-TMPRSS2 cells are utilized to culture and quantitate 3CLpro S301P virus, which is rescued using VeroE6-TMPRSS2 cells. FIG. 3B. Replication kinetics of 3CLpro S301P recombinant virus. Both recombinant WT and mutant viruses replicate at comparable rate, 3CLpro S301P showed slight attenuation in growth rate. FIG. 3C. Microscopic images of recombinant WT and 3CLpro S301P mutant virus in the presence of compound 172 and percentages of CPE of each image. VeroE6-TMPRSS2 is infected with either virus at MOI 0.01 and treated with compound 172 at different concentrations ranging from 20P M to 0.625 μM. Representative images of CPE formation are captured at 48 hpi. Scale bar=100 μm. The experiment is performed in triplicate and repeated twice for confirmation. The CPE severity in each group is scored and compared by two-way ANOVA. FIG. 3D. Plaque formation assay of recombinant WT and 3CLpro S301P virus under the treatment of compound 172, Pelitinib and Nirmatrelvir, respectively. Antiviral IC₅₀ against of each drug compound against WT and mutant viruses are plotted by GraphPad. ***P<0.001; **P<0.01; *P<0.05.

FIG. 4A-4I. Mechanistic investigation of compound 172. FIG. 4A. FRET-based protease activity assay. Recombinant WT and S301P 3CLpro are expressed and purified. Compound 172 is diluted from 100 μM to 6.26 μM and incubated with 1.25 μM WT or S301P 3CLpro at RT for 30 minutes. 50 μM fluorophore-conjugated substrate is added into the mixture after 20 minutes as previously established. 200 nM GC376 is added as a positive control. Both experiments are performed in triplicate. Statistical analysis by Student's T-test: ***P<0.001; **P<0.01; *P<0.05. FIG. 4B-4C. Surface plasmon resonance (SPR) spectroscopy of compound 172 with WT and S301P 3CLpro. The Biacore T-200 machine is used to conduct SPR spectroscopy. 40 μg/mL of protease is immobilized on a Series S Sensor Chip CM5. Compound 172 is added to the chip at gradient concentrations ranging from 100 μM to 3.125 μM. Cytiva software is used to generate the association-dissociation graph and the dissociation constant (KD). FIG. 4D-4E.Michaelis-menton inhibition kinetics of compound 172 and Nirmatrelvir. 1.25 μM WT 3CLpro is incubated with compound 172 or Nirmatrelvir in a gradient of concentrations at RT for 30 minutes, followed by addition of fluorophore-conjugated substrate in a range of concentrations and measured in fluorescence (Excitation: 365 nm, Emission: 500-550 nm) in 20 minutes as initial velocity. FIG. 4F. Analytical ultracentrifugation (AUC) for WT 3CLpro with compound 172. 600 μg/mL WT 3CLpro is incubated with 20 μM compound 172 or 0.2% DMSO before this sedimentation velocity experiment. The main peaks for 3CLpro monomer and dimer showed a sedimentation coefficient of 3.3 S and 4.6 S respectively. Two independent experiments are performed. Statistical analysis by Student's T-test: ***P<0.001; **P<0.01; *P<0.05. FIG. 4G-4H. Intermolecular interaction of pelitinib and compound 172 to 3CLpro. Monomers are shown in cyan and green cartoon representation, respectively. Interacting residues of 3CLpro within 3.5 Å of compound are shown in magenta sticks and labelled accordingly. FIG. 4I. List of Loewe's additivity (LA) indices calculated with the IC50 values of each compound combination. LA<1, synergism; LA=1, independent; LA>1, antagonism. 172 concentrations that lowered Nirmatrelvir IC50 are highlighted in rows 5 and 6. This experiment is conducted in triplicates.

FIG. 5A-5G. In vivo activity of compound 172. FIG. 5A. Permeability of compound 172 in Caco-2 cells and metabolic stability of 172 in mouse and human Liver microsomes. FIG. 5B. In vivo pharmacokinetic profiling of compound 172 in a BALB/c mouse model (n=3). FIG. 5C. Schematics of compound 172 administration in a golden Syrian hamster model. Two groups of hamsters (n=4) are given intranasal inoculations of 1000 PFU/animal of WT SARS-CoV-2. The treatment group (n=4) received compound 172 (1 mg/kg) or nirmatrelvir (200 mg/kg) via intraperitoneal (IP) injection, or pelitinib (10 mg/kg) via oral gavage. The vehicle group (n=4) is given 5% DMSO in 20% SBE-β-CD/0.9% saline. On the third day following the infection, the hamsters are euthanized for both viral yield and histopathological examination. FIG. 5D. Hamsters lung and nasal turbinate viral yield are determined by RT-qPCR. Statistical analysis by one-way ANOVA: ***P<0.001; **P<0.01; *P<0.05. FIG. 5E. Representative images of H&E-stained and IF-stained lung tissue section from hamster treated as indicated. Scale bars: 100 μm. FIG. 5F. Schematics of compound 172 administration on K18-hACE2 mice model. K18-hACE2 mice are intranasally inoculated with either Alpha (B.1.1.7, for survival rate) or Omicron (BA.5, for viral load detection) at 200 PFU/animal or 10,000 PFU/animal (n=5 for the survival study and n=4 for the viral load study) respectively. The treatment group is given compound 172 (50 mg/kg) dissolved in a solution of 20% SBE-β-CD/0.9% saline, administered once daily via IP injection. The vehicle group is given 5% DMSO in 20% SBE-β-CD/0.9% saline using the same treatment regimen. The positive control group is given Nirmatrelvir (200 mg/kg) dissolved in a solution of 20% SBE-β-CD and 0.9% saline using the same treatment regimen. The mice in the survival study are monitored daily, and drugs are administered until they reached the humane endpoint or died. The mice in the viral load study are euthanized on the third day after the infection, and their lungs and nasal turbinate are collected for viral load quantification. FIG. 5G. Mouse survival rate (upper panel) and daily body weight changes (lower panel) of K18-hACE2 mice. Comparison of survival rates between groups are analysed using Log-rank (Mantel-Cox) tests and that of body weight using two-way ANOVA. (FIG. 5H) K18-hACE2 mouse lung and nasal turbinate viral titre determined by standard plaque assay. Statistical analysis by one-way ANOVA: ***P<0.001; **P<0.01; *P<0.05.

FIG. 6. SMART™ phenotype-based 3-tier screening methodology. Primary screening is conducted on VeroE6 cells based on CPE inhibition and 168 compounds are shortlisted out of 50,213 raw compounds from SMART™ library. Secondary screening is performed on A549-TMPRSS2-ACE2 cells and 40 out of 168 compounds exhibited >2-log viral load reduction at one or more concentrations when compared to DMSO control. Tertiary screening is performed by Plaque Reduction assay and five compounds are confirmed to reduce infectious particle titer with relatively low cytotoxicity.

FIG. 7. Secondary screening results. A549-TMPRSS2-ACE2 cells are challenged by SARS-CoV-2 WT D614G virus at MOI 0.01. After 48 hpi, the viral load in the cell supernatant is measured by RT-PCR. The viral load is represented on a color scale in log 10 scale. Compounds that exhibited more than a 2-log 10 reduction in viral load/mL at one or two concentrations when compared to the DMSO control are identified as secondary hit compounds. 40 compounds are found to meet this criterion and are indicated by red arrows.

FIG. 8. Immunofluorescence staining of SARS-CoV-2 nucleocapsid (N) antigen. N antigen labelled in green and cell nuclei labelled in blue. Vero-TMPRSS2 cells are fixed and stained after SARS-CoV-2 infection at MOI 0.01 and treated with compounds (10M) for 48 hours. Scale bar=100 μm.

FIG. 9. MTT cytotoxicity assays of SMART™ finalized hits compounds on VeroE6 cells. VeroE6 cells are treated with compounds of interest diluted from 100 μM to 0.78 μM in a 2-fold interval and incubate for 72 hours.

FIG. 10A.Schematics of compound 172 escape mutant generation. 4×10⁵ Vero-TMPRSS2 cells per well is infected by MOI 1 SARS-CoV-2 WT D614G and treated with 5 μM of compound 172, and then incubated for 48 hours until 50% of the cells exhibited cytopathic effects. Next, 100 μL of passage 1 is transferred to a new plate of Vero-TMPRSS2 cells, and after 1 hour, the medium is replaced with 6 μM of compound 172. This process is repeated with increased drug concentration and reduced MOI until passage 6, which showed 50% CPE formation in less than 48 hours in the presence of 8 μM compound 172. Passage 6 is then divided into aliquots and the viral titer is measured using plaque assay. The resulting escape phenotype is confirmed through RT-PCR analysis, and the genotype is analyzed using Nanopore sequencing. This schematic is created with Biorender.com. FIG. 10B. The S301P point mutation is verified by traditional Sanger sequencing. Red box indicates the substitution of serine residue by proline at the 301st position of the 3CL-protease. FIG. 10C. The S301P point mutation in the bacterial artificial chromosome (BAC) plasmid and rescued virus are verified by traditional Sanger sequencing. Blue box indicates the difference between WT and mutant plasmid. Red box indicates the substitution of serine residue by proline at the 301st position of the 3CL-protease.

FIGS. 11 and 12. Antiviral synergy and predicted binding stability of compound 172. FIG. 11. Compound 172-Nirmatrelvir drug synergism checkboard assay result. A549-TMPRSS2-ACE2 cells are treated with compound 172 and Nirmatrelvir mixture in a checkerboard manner. The cells are challenged by WT SARS-CoV-2 at MOI 0.1. Cell viability is measured by CellTiter Glo reagent (Promega, USA) at 48 hpi. EC50 values of each combination of compound 172 and Nirmatrelvir are computed and plotted using GraphPad Prism software. The line connecting the EC50 of each compound alone is coined “isobole”, any points within the isobole are considered synergism and beyond are considered antagonism.

FIG. 12. Ligand RMSD analysis of three different 10 ns molecular dynamics (MD) simulations of 172 bound to 3CLpro.

FIG. 13A-13B. Sedimentation distribution profile of AUC experiments. FIG. 13A. The main peaks for 3CLpro monomer and dimer displayed a sedimentation coefficient of 3.3S and 4.6S respectively. The 600 μg/ml freshly purified WT 3CLpro is incubated with 20 μM 172 or 0.2% DMSO for at least 2 h on ice prior to conducting this assay. After incubating with 172, the contribution of dimer reduced about 10%, while the opposite is observed for monomers. FIG. 13B. Binding affinity measurement comparing 3CLpro monomer and dimer. The dimer form is expression and purified as described in the methodology. A monomer form is engineered by extension at its N-terminus that hinders dimerization, whereas retains the catalytic site and dimerization interface. The binding affinity is measured by Bio-Layer Interferometry analysis.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### DETAILED DESCRIPTION

Given the complex life cycle of SARS-CoV-2 which involves multiple steps, there are potentially new antiviral pathways that remain unexplored. Chemical genetics is one of the approaches to reveal novel antiviral pathways. It is the study of functions of genes by disruption with small chemical molecules (29, 30). Chemical genetics has benefits over traditional genetics in the discovery of novel druggable targets. Traditional reverse genetics often relies on altering gene expression, which may not fully capture the complexity of protein function, such as post-translational modifications or protein interactions (29, 30). Additionally, some proteins are essential for normal cellular function, knocking them out could be detrimental.

Chemical genetics can be divided into two categories: forward and reverse chemical genetics (30). Forward chemical genetics involves studying the phenotypic changes caused by small molecules, which can lead to the discovery of novel drug targets (30). Reverse chemical genetics, on the other hand, requires a defined cellular target, followed by studying its function using small molecules (30). This approach can be used to identify new small molecules that can achieve a desired phenotype. 50,213 raw compounds from the SMART™ Library are screened from a forward chemical genetics direction, to determine their ability to inhibit cytopathic effect on VeroE6 cells. Secondary screening is then performed on A549-TMPRSS2-ACE2 cells to identify compounds with dose-dependent antiviral activity. The antiviral properties of the screened compounds are validated using plaque reduction assay and MTT cytotoxicity assay.

Ultimately, five out of 50,213 compounds are confirmed to have anti-SARS-CoV-2 activity in vitro. Among these, 10-(4-methylphenyl)-7-phenyl-6,7,8,10-tetrahydro-5H-indeno[1,2-b]quinoline-9,11-dione (designated compound 172), exhibited the highest selectivity index (SI), inhibited several SARS-CoV-2 variants of concern (VOC) and multiple human coronaviruses including MERS-CoV (Middle East respiratory syndrome coronavirus), SARS-CoV (Severe Acute Respiratory Syndrome coronavirus), and HCoV-229E (Human coronavirus 229E). Importantly, compound 172 also demonstrated antiviral activity in both Golden Syrian Hamster and K18-hACE mice models. Interestingly, mechanistic studies revealed that compound 172 targets a novel allosteric site on 3CLpro domain III and interferes with protein dimerization. Additionally, compound 172 can achieve drug synergism with Nirmatrelvir, an active compound in Paxlovid (16, 31), at nanomolar concentrations.

### I. Defintions

The terms “individual”, “host”, “subject”, and “patient” are used interchangeably herein, and refer to a mammal, including, but not limited to, humans, rodents, such as mice and rats, and other laboratory animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions

### A. Compound 172 and Pharmaceutically Acceptable Salts Thereof

The disclosed methods include administering to a subject in need thereof, an effective amount of a mall-molecular allosteric protease inhibitor, 10-(4-methylphenyl)-7-phenyl-6,7,8,10-tetrahydro-5H-indeno[1,2-b]quinoline-9,11-dione (compound 172, having formula (I) as below), or a derivative thereof.

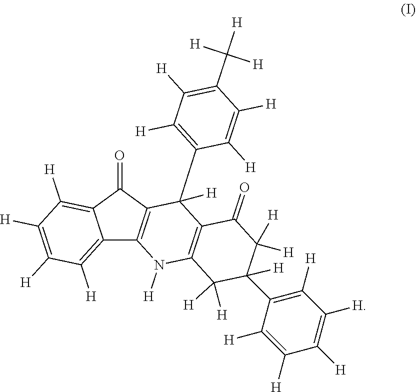

The term “derivative” does not mean that the derivative is synthesized from the parent compound either as a starting material or intermediate, although this may be the case. The term “derivative” can include salts, prodrugs, or metabolites of the parent compound.

Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include replacing one or more amino substituents or hydrogen groups with substituted or unsubstituted alkyl, aminoalkyl, aryl, or heteroaryl groups having from 1 to 30 carbon atoms.

Examples of pharmaceutically acceptable salts include, but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, MD, 2000, p. 704; and “Handbook of Pharmaceutical Salts: Properties, Selection, and Use,” P. Heinrich Stahl and Camille G. Wermuth, Eds., Wiley-VCH, Weinheim, 2002.

In some forms, the composition further comprises nirmatrelvir, which displays drug synergism with compound 172.

The compounds identified herein using virtual ligand screening can be used as a starting compound or intermediate compound to produce a final compound with antiviral activity. The compounds identified herein can be modified to increase bioavailability, increase half-life in the blood stream, increase solubility, increase hydrophilicity, increase hydrophobicity, or a combination thereof using conventional techniques.

The compounds can be modified to incorporate polar functional groups, such as the alcohol, amine, amide, carboxylic acid, sulfonic acid and phosphate groups, which either ionize or are capable of relatively strong intermolecular forces of attraction with water (hydrogen bonding), usually resulting in analogues with an increased water solubility. Acidic and basic groups are particularly useful, since these groups can be used to form salts, which would give a wider range of dosage forms for the final product. However, the formation of zwitterions by the introduction of either an acid group into a structure containing a base or a base group into a structure containing an acid group can reduce water solubility. Introduction of weakly polar groups, such as carboxylic acid esters, aryl halides and alkyl halides, will not significantly improve water solubility and can result in enhanced lipid solubility.

The incorporation of acidic residues into the compound is less likely to change the type of activity, but it can result in the analogue exhibiting hemolytic properties. Furthermore, the introduction of an aromatic acid group usually results in anti-inflammatory activity, whilst carboxylic acids with an alpha functional group may act as chelating agents. Basic water solubilizing groups have a tendency to change the mode of action, since bases often interfere with neurotransmitters and biological processes involving amines. However, their incorporation does mean that the analogue can be formulated as a wide variety of acid salts. Non-ionizable groups do not have the disadvantages of acidic and basic groups.

Groups that are bound directly to the carbon skeleton of the lead compound by less reactive C—C, C—O and C—N bonds are likely to be irreversibly attached to the lead structure. Groups that are linked to the compound by ester, amide, phosphate, sulfate and glycosidic bonds are more likely to be metabolized from the resulting analogue to reform the parent compound as the analogue is transferred from its point of administration to its site of action. Compounds with this type of solubilizing group are acting as prodrugs and so their activity is more likely to be the same as the parent compound. However, the rate of loss of the solubilizing group will depend on the nature of the transfer route, and this could affect the activity of the drug.

To preserve the type of activity exhibited by the compound, a water solubilizing group should be attached to a part of the structure that is not involved in the drug-receptor interaction. Consequently, the route used to introduce a new water solubilizing group and its position in the lead compound will depend on the relative reactivities of the compound and the rest of the molecule. Examples of water solubilizing structures and the routes used to introduce them into the lead structures. O-alkylation, N-alkylation, O-acylation and N-acylation reactions are used to introduce both acidic and basic groups. Acetylation methods use both the appropriate acid chloride and anhydride.

Examples of water solubilizing structures and the routes used to introduce them into lead compounds include but are not limited to phosphate acid halides for introducing phosphate groups into compounds. Structures containing hydroxy groups have been introduced by reaction of the corresponding monochlorinated hydrin and the use of suitable epoxides. Sulphonic acid groups may be introduced by either direct sulfonation or the addition of bisulfite to reactive C═C bonds.

Nirmatrelvir is an antiviral, which acts as an orally active 3C-like protease inhibitor.

### B. Formulations

Compound 172 can be formulated for enteral, parenteral, or pulmonary administration. In a preferred embodiment, the peptide is formulated for pulmonary administration.

Compound 172 can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

Compound 172 disclosed herein can also be formulated for use as a disinfectant, for example, in a hospital environment.

### 1. Pulmonary Formulations

In one embodiment, compound 172 is formulated for pulmonary delivery, such as intranasal administration or oral inhalation.

The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions including low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the sub epithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm3, porous endothelial basement membrane, and it is easily accessible.

Carriers for pulmonary formulations can be divided into those for dry powder formulations and for administration as solutions. Aerosols for the delivery of therapeutic agents to the respiratory tract are known in the art. Aerosols can be produced using standard techniques, such as ultrasonication or high-pressure treatment. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

Solvents that are low toxicity organic (i.e. nonaqueous) class 3 residual solvents, such as ethanol, acetone, ethyl acetate, tetrahydofuran, ethyl ether, and propanol may be used for the formulations. The solvent is selected based on its ability to readily aerosolize the formulation. The solvent should not detrimentally react with compound 172. An appropriate solvent should be used that dissolves the compounds or forms a suspension of compound 172. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of compound 172 in the lungs and that the excipients that are present are present in amount that do not adversely affect uptake of compound 172 in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

### 2. Parenteral Formulations

Compound 172 can be formulated for parenteral administration.

For example, parenteral administration may include administration to a patient intravenously, intradermally, intraarterially, intraperitoneally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating compound 172 in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### (a) Controlled Release Formulations

The parenteral formulations described herein can be formulated for controlled release including immediate release, delayed release, extended release, pulsatile release, and combinations thereof.

i. Nano- and Microparticles

For parenteral administration, compound 172, and optional one or more additional active agents, can be incorporated into microparticles, nanoparticles, or combinations thereof that provide controlled release of the disclosed compound 172 and/or one or more additional active agents. In embodiments wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, compound 172 and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

ii. Method of Making Nano- and Microparticles

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### (b) Injectable/Implantable Formulations

Compound 172 can be incorporated into injectable/implantable solid or semi-solid implants, such as polymeric implants. In one embodiment, compound 172 is incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, compound 172 can be incorporated into a polymer matrix and molded, compressed, or extruded into a device that is a solid at room temperature. For example, compound 172 can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the peptide from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

### 3. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. IN embodiments where the formulation is for oral administration involving transit through the gastrointestinal tract, the formulation is preferably coated to protect the peptide from gastrointestinal enzymes.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### (a) Controlled Release Enteral Formulations

Oral dosage forms, such as capsules, tablets, solutions, and suspensions, can for formulated for controlled release. For example, compound 172 and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, compound 172 and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, compound 172 and optional one or more additional active agents are formulated into a sold oral dosage form, such as a tablet or capsule, and the solid dosage form is coated with one or more controlled release coatings, such as a delayed release coatings or extended release coatings. The coating or coatings may also contain compound 172 and/or additional active agents.

i. Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename EUDRAGIT T®. In further preferred embodiments, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT T® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

ii. Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### III. Methods of Use

The disclosed methods are based on studies showing that P9R exhibits very broad-spectrum antiviral activities against the enveloped SARS-CoV-2, exhibited the highest selectivity index (SI) among tested compounds, inhibiting several SARS-CoV-2 variants of concern (VOC) and multiple human coronaviruses including MERS-CoV (Middle East respiratory syndrome coronavirus), SARS-CoV (Severe Acute Respiratory Syndrome coronavirus), and HCoV-229E (Human coronavirus 229E). Importantly, compound 172 also demonstrated antiviral activity in both Golden Syrian Hamster and K18-hACE mice models.

Accordingly, methods are provided for treating a subject infected with a coronavirus, particularly, an enveloped coronavirus, by administering the subjected a formulation containing an effective amount of the disclosed peptides, to ameliorate one or more symptoms associated with the viral infection. In a preferred embodiment, the treatment is effective to inhibit viral replication in the subject. The subject can be treated with the disclosed peptides by administering an effective amount of the peptide to the subject, enterally, by pulmonary or nasal administration, or parenterally (intravenously, intradermally, intraarterially, intraperitoneally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

The following non-limiting examples further explain the disclosed and claimed compositions and methods.

### EXAMPLES

### Example 1: Materials and Methods

### Chemical Reagents

SMART™ Library purchased from ChemDiv (San Diego, USA) contains a total of 50,213 structurally diverse synthetic raw compounds. The whole library is formatted in 167 384-well plates of 10 mM stock concentration dissolved in DMSO per well. The library is kept at −80° C. for storage. Remdesivir. GC-376 and Nirmatrelvir are purchased from MedChemExpress (NJ, USA). FRET-based protease activity substrate: Dabcyl-KTSAVLQSGFRKM-E(Edans)-NH2 is purchased from Bachem Bioscience (Bubendorf, Switzerland). CellTiter Glo® cell viability assay kit is purchased from Promega USA.

### Cell and Virus Culture

Multiple cell lines are used in this study: VeroE6, Vero-TMPRSS2, A549-TMPRSS2-ACE2, Huh7, U251 and HK2. VeroE6, U251, Huh7 and HK2 are maintained in DMEM supplemented with 5-10% fetal bovine serum (FBS) and 1% penicillin-streptomycin (PS). Vero-TMPRSS2 is maintained in 10% FBS DMEM supplemented with 1% PS and lmg/mL G-418 for marker selection. A549-TMPRSS2-ACE2 is maintained in 10% FBS DMEM supplemented with 1% PS, 0.5 μg/mL Puromycin and 300 μg/mL Hygromycin for marker selection. All cell lines are passaged at least once per week.

Several SARS-CoV-2 variants are used in this study: Wild Type (strain HKU-001a) (GenBank: MT230904), strain B.1.1.7 (Alpha) (GenBank: OM212469), strain B.1.617.2 (Delta) (GenBank: OM212471), Omicron BA.1, and Omicron BA.5 (GenBank: OM212472). These variants are isolated from patients with laboratory confirmed COVID-19 in Hong Kong (32). Virus expansion and propagation are conducted as previously described (32). The resulting virus stocks are stored at −80° C. MERS-CoV (EMC/2012) is kindly provided by Ron Fouchier (Erasmus Medical Center, the Netherlands). Archived clinical strains of SARS-CoV-1 (GZ50 strains, GenBank: AY304495), and HCoV-229E are obtained from the Department of Microbiology, The University of Hong Kong. Experiments involving live SARS-CoV-2 virus are performed in the BSL-3 facility located in Block T of Queen Mary Hospital.

### Primary Screening of SMAR™ Library by CellTiter-Glo® Assay

VeroE6 cells are dispensed onto 384-well plates at a density of 5000 cells per well using a liquid dispenser. Selected SMART™ stock plates are diluted 20-fold using the Apricot S1 automatic 384 format liquid handling system (Apricot Designs, USA), and 0.5 μl of the diluted stock is added to each well using the same system. A positive control, Remdesivir at 10 μM, is added to column 23. The cells and compounds are co-incubated overnight at 37° C. with 5% CO2 before being transferred to the BSL-3 facility for infection with SARS-CoV-2 WT strain D614G at a multiplicity of infection (MOI) of 0.01 in 10 μl per well, except for column 1 and 24 which served as mock infections. The final compound concentration is 5 μM. After a 3-day incubation at 37° C. with 5% CO₂, CellTiter Glo® reagent is added to each well, and cell viability is measured using a plate reader located inside the BSL-3 facility. The primary screening is performed in duplicate, and compounds that demonstrated >60% cell viability in both duplicates are selected for secondary screening. The cell viability data is analysed using a self-developed R studio script.

### Secondary Screening by RT-qPCR

The day before infection, A549-TMPRSS2-ACE2 cells are seeded onto 96-well plates at a density of 20,000 cells per well. On the following day, the plates are transferred to the BSL-3 facility for infection with SARS-CoV-2 strain D614G at MOI 0.01. After a 1-hour adsorption period, the cell supernatant is removed, and the cells are washed once with PBS before being treated with primary hit compounds that had been prepared in advance at concentrations of 20 μM, 5 μM, and 1.25 μM. Following a 2-day incubation period at 37° C. with 5% CO2, the cell supernatant is collected and lysed using RLT lysis buffer. RNA extraction is performed using the RNeasy kit (Qiagen, Germany) according to the manufacturer's protocol. The extracted RNA is then quantified using the Takara One-Step TB Green® PrimeScript™ RT-QPCR kit II (Takara, Japan) and SARS-CoV-2 RdRp/Hel gene primers as described previously (32). Primary hit compounds that resulted in more than a 2-log 10 viral load reduction in one or more concentrations are considered as secondary hits and would be validated by Plaque Reduction Assay in tertiary screening.

### Plaque Reduction Assay

Briefly, compounds of interest are prepared in 2× gradient concentrations using 2% FBS/2% PS DMEM and mixed with LMP-agarose at a 1:1 ratio after a 1-hour virus adsorption period. The compound-agarose mixture is then overlayed onto cells, and the plates are inverted and placed in a 37° C./5% CO2 incubator for 3 days. A solution of 10% formalin in PBS is used to fix the cells, and a 0.25% solution of crystal violet is used to stain the plaques. The GraphPad Prism software is used to compute an IC50 value for each compound that demonstrated a reduction in plaque activity without significant cell toxicity.

### MTT Cytotoxicity Assay

Briefly, cells are seeded onto a 96-well plate at a density of 20,000 cells per well one day before treatment. On the following day, compounds of interest are added to the cells in gradient concentrations and incubated at 37° C. with 5% CO2 for either 48 or 96 hours. After medium removal, 100 μL of 1×MTT solution in plain DMEM is added to the cells and incubated at 37° C. with 5% CO2 for 3-4 hours. Then, MTT lysis buffer (10% SDS/10 mM HCl) is added to the cells and incubated at room temperature for 3-4 hours. Cell viability is measured in absorbance (OD595) using a plate reader (Glomax, Promega).

### SARS-CoV-2 N Antigen Expression Assay

After infecting VeroE6 cells with SARS-CoV-2 for one hour at a 37° C./5% CO2 incubator, the medium is removed and replaced with 1% FBS 1% PS DMEM containing the compound of interest. After 3 days post-infection (dpi), the medium is removed, and the cells are fixed with a 10% formaldehyde solution in PBS for 15 minutes. Following this, the formaldehyde is removed, and the cells are rinsed briefly with Virkon before being transferred to a BSL-2 laboratory for IF staining. The IF staining process involved adding 0.1% Triton X-100 in PBS to permeabilize the cells. Next, 2% bovine serum albumin (BSA) in PBS is used to incubate with the cells for one hour at room temperature (RT) to reduce non-specific bindings of the primary antibody. Then, an in-house rabbit antiserum against SARS-CoV-2 N protein, diluted at a ratio of 1:200 in 1% BSA in PBS, is added to the cells and incubated at 4° C. overnight. The cells are washed twice with PBS-T (0.05% Tween 20 in PBS) after removing the primary antibody, followed by the addition of Alexa Fluor secondary antibody (ThermoFisher Scientific, USA) at 1:500 dilution and incubation for 1-2 hours at RT. The cells are then labeled with 4′,6-diamidino-2-phenylindole (DAPI) nucleic acid stain (ThermoFisher Scientific, USA) to label cell nuclei and mounted with the Diamond Prolong Antifade mountant (ThermoFisher Scientific, USA). Finally, the stained cells are observed under a fluorescent microscope to determine the antigen's intensity.

### Viral Load Reduction Assay by RT-qPCR

Viral load reduction assay is performed by quantitative reverse transcription-polymerase chain reaction (RT-qPCR) is used with slight modifications from a previously described protocol (33). RNA is extracted from the culture supernatants of CoV-infected cell lines, as described earlier, using the RNeasy extraction kit (Qiagen, Germany). Reverse transcription is performed using the Transcriptor First Strand cDNA Synthesis Kit from Roche (Basel, Switzerland) with oligodT primers. To determine the virus genome copies, qPCR is performed using the LightCycler 480 SYBR Green I Master Mix from Roche with specific primers. The virus genome copies in the supernatant samples are quantified with a standard using the following specific primers: hCoV-229E_Forward: CTACAGATAGAAAAGTTGCTTT (SEQ ID NO:1); hCoV-229E_Reverse: GGTCGTTTAGTTGAGAAAAGT (SEQ ID NO:2); SARS-CoV(-2)_Forward: CGCATACAGTCTTRCAGGCT (SEQ ID NO:3); SARS-CoV(-2)_Reverse: GTGTGATGTTGAWATGACATGGTC (SEQ ID NO:4); MERS-CoV_Forward: GGGTGTACCTCTTAATGCCAATTC (SEQ ID NO:5): MERS-CoV_Reverse: TCTGTCCTGTCTCCGCCAAT (SEQ ID NO:6).

### 1.9 Generation and RT-qPCR Validation of Escape Mutant

To investigate the antiviral mechanism of compound 172, an escape mutant is generated. Vero-TMPRSS2 cells are seeded onto a 12-well plate at a density of 4×10⁵ cells per well one day before infection to generate passage 1. On the next day, SARS-CoV-2 WT D614G is inoculated into the cells at MOI 1 in the presence of 5 μM of compound 172 or 0.05% DMSO (control). After 48 hours of incubation at 37° C./5% CO2, around 50% CPE formation is observed. The supernatant of passage 1 is transferred to a new plate of Vero-TMPRSS2 cells and absorbed for one hour at 37° C., and the remaining of passage 1 is stored at −80° C. After one hour, the cell medium is replaced with 6 μM of compound 172 or 0.06% DMSO, and the plate is incubated at 37° C./5% CO2. This procedure is repeated with decreasing MOI and increasing drug concentration. At passage 6, CPE formation is observed in the presence of 8 μM compound 172, and it is collected for plaque quantification, escape mutant validation, and Nanopore sequencing.

The escape mutant is validated using the RT-qPCR method. Vero-TMPRSS2 cells are seeded onto a 96-well plate at a density of 20,000 cells per well one day before infection. The cells are then infected with virus from passage 6 and passage 0 (strain D614G) at MOI 0.01, and treated with compound 172 in gradient concentration after one hour of virus adsorption. After 48 hours post-infection (hpi), the cell supernatant is collected, and RNA is extracted using the RNeasy kit from Qiagen (Germany) following the manufacturer's protocol. The viral RNA copy is measured using the Takara One-Step TB Green® PrimeScript™ RT-qPCR kit II from Takara (Japan) with SARS-CoV-2 RdRp/Hel gene as mentioned previously.

### 1.10 Nanopore Sequencing of Escape Mutant

Nanopore sequencing technology (Oxford Nanopore Technologies, UK) is used to perform whole genome sequencing of SARS-CoV-2, as previously described (34). RNA is extracted from passage 6 using the RNeasy kit (Qiagen, Germany) and then reverse transcribed into cDNA using SuperScript™ IV reverse transcriptase (ThermoFisher Scientific, USA) according to the manufacturer's protocol. PCR is performed using the ARTIC nCov-2019 V3 Panel (IDT, USA) and Q5® Hot Start High-Fidelity 2× Master Mix (New England Biolabs, United States), as per the manufacturer's protocol. PCR product cleanup is conducted using 1× Agencourt AMPure XP (Beckman Coulter, USA). End-prep, barcode ligation, and sequencing adaptor ligation are performed according to the PCR tiling of SARS-CoV-2 virus with Native Barcoding Expansion 96 protocol (Version: PTCN_9103_v109_revH_13jul.2020). The library is loaded and sequenced on MinION with R9.4.1 flow cells for 48 hours. Nanopore sequencing data are analyzed following the Artic Network nCoV-2019 novel coronavirus bioinformatics protocol with minor modifications (35).

Generation of Recombinant Mutant Virus from Bacterial Artificial Chromosome (BAC)

An infectious molecular clone of SARS-CoV-2 on a Bacterial Artificial Chromosome (BAC) is used. The 3CLpro S301P substitution is introduced into the clone using k-Red-mediated homologous recombination, as we previously described (36). Firstly, homologous recombination is carried out to insert a galK expression cassette, which is amplified using 50-bp 3CLpro-flanking homology arms to bind to plasmid pMOD4-galK-G (5′-3′ Forward: GTAGTGCTTTATTAGAAGATGAATTTACACCTTTTGATGTTGTTAGACAACCTGTT GACAATTAATCATCGGCA ((SEQ ID NO:7); 5′-3′ Reverse: GTGGTGTGTACCCTTGATTGTTCTTTTCACTGCACTTTGGAAAGTAACACTCAGCA CTGTCCTGCTCCTT (SEQ ID NO:8)). The galK cassette is introduced into the site of interest by electroporation into competent E. coli SW105 strain. Electroporated bacterial cells are plated onto M63 minimal agar supplemented with galactose for 3 days at 32° C. The galK-bearing clone is isolated and purified, and then subjected to a second round of recombination. Secondly, annealed oligonucleotides bearing the 3CLpro S301P mutation ordered from Integrated DNA Technologies (IDT) are electroporated to substitute the galK cassette (5′-3′ Forward: GTAGTGCTTTATTAGAAGATGAATTTACACCTTTTGATGTTGTTAGACAATGCCC AGGTGTTACTTTCCAAAGTGCAGTGAAAAGAACAATCAAGGGTACACACCAC (SEQ ID NO:9); 5′-3′ Reverse: GTGGTGTGTACCCTTGATTGTTCTTTTCACTGCACTTTGGAAAGTAACACCTGGGC ATTGTCTAACAACATCAAAAGGTGTAAATTCATCTTCTAATAAAGCACTACSEQ ID NO:10)). The electroporated-bacterial cells are plated onto M63 minimal agar supplemented with 2-deoxy-galactose to remove the galK-bearing clone. The successful clone is screened using PCR amplification flanking the 3CLpro site of interest, and the point mutation is confirmed through Sanger Sequencing. Purified BAC bearing the 3CLpro S301P mutation is extracted using the PureLink™ HiPure BAC Buffer Kit (ThermoFisher Scientific, USA).

Recombinant virus rescue is performed in Vero-TMRPSS2 cells. 6 μg of BAC is transfected into the cells using Lipofectamine 3000 according to the manufacturer's instructions, and then transferred into a BSL-3 facility. After 6 hours, the BAC-containing medium is replaced with serum-free DMEM. The cells are monitored for cytopathic effect over the course of 3 days. Upon confirmation of cytopathic effect, virus-containing supernatant is clarified and harvested. SARS-CoV-2 3CLpro S301P is further passaged in VeroE6-TMPRSS2 cells, and the viral titers are determined using plaque assay.

### 1.12 Validation of Antiviral Resistance Recombinant Mutant Virus

The antiviral resistance phenotype of the recombinant mutant virus is confirmed using three methods: phenotypic observation, RT-qPCR, and plaque reduction assay. Phenotypic validation involved observing CPE formation. Vero-TMRPRSS2 cells are seeded onto a 96-well plate at a density of 20,000 cells/well one day before infection. On the following day, the cells are infected with recombinant WT and 3CLpro S301P mutant viruses at an MOI of 0.01, and the cell medium is replaced with compound 172 in gradient concentration after one hour of virus adsorption. After 48 hpi, CPE formation is observed under an electronic light microscope, and photos are taken in the BSL-3 facility. Cell supernatant is collected and RNA extraction is performed using the RNeasy kit from (Qiagen, Germany) following the manufacturer's protocol. The SARS-CoV-2 viral RNA of both recombinant WT and mutant viruses is measured by RT-qPCR using the Takara One-Step TB Green® PrimeScript™ RT-qPCR kit II (Takara, Japan), with SARS-CoV-2 RdRp/Hel gene primers as mentioned. Plaque reduction assay is also used to validate the antiviral resistance phenotype of the recombinant mutant virus as previously described.

### Drug Synergism Checkerboard Assay

Compound 172 and Nirmatrelvir are prepared in a gradient of 4× concentrations, mixed in a checkerboard pattern at a 1:1 ratio, and added to A549-TMPRSS2-ACE2 cells are seeded onto 96-well plates one day before. On the next day, Wild type SARS-CoV-2 is then added to the cells at a 1:1 volume ratio with a MOI of 0.1. After 48 hours post-infection (hpi), CellTiter Glo® reagent is added to the cells, and cell viability is measured in luminance using a plate reader in a BSL-3 facility. The cell viability is normalized and fitted into a non-linear regression model using GraphPad Prism, and an IC50 value is calculated for each compound with the corresponding partner compound concentration. The paired IC50 values with the corresponding partner compound concentrations are plotted as a scatterplot for data visualization. The paired values are also fitted into the Loewe's additivity (LA) equation, as follows: [A]/IC50(A)+[B]/IC50(B)=1.

### Expression and Purification of SARS-CoV-2 WT and Mutant 3CL-Protease

Recombinant SARS-CoV-2 WT 3CLpro and mutant (S301P) using the reference Wuhan-Hu-1 (GenBank ID: YP_009724390.1) are codon-optimized and cloned into pET28b+ for E. coli BL21 expression as previously described (37). A 6×His tag is added to the N-terminal of each protein construct for Ni-NTA purification. To induce recombinant 3CLpro production, the E. coli subculture is grown until reaching an OD600 absorbance of 0.6, and then induced with 0.5 mM Isopropyl B-D-1-thiogalactopyranoside (IPTG) at 25° C. for 16 hours with agitation. After overexpression, the bacterial pellet is collected, lysed with 0.1% Triton X-100 in PBS, and stored at −80° C. overnight. The total proteins are released from the lysate through sonication, followed by centrifugation at 13,000 g/4° C. for 30 minutes. The solution is passed through the Ni-NTA column twice to bind, washed with 500 mL washing buffer (50 mM NaH2PO4, 300 mM NaCl, 40 mM imidazole, pH 8.0) at a controlled and steady flow rate, then eluted with 10 mL elution buffer (50 mM NaH2PO4, 300 mM NaCl, 250 mM imidazole; pH 8.0) into fractions. The purity of each fraction is analyzed by SDS-PAGE followed by Coomassie blue staining. The concentration of the purified 3CLpro is determined using the Bradford Assay.

### Fluorescence-Based Protease Activity Assay

A fluorescent-resonance-energy-transfer (FRET)-based protease activity assay is developed to investigate the inhibitory effects of compound 172 on the enzymatic activity of WT and mutant 3CLpro. Compound 172 is prepared in 50× gradient concentration in DMSO, while substrate (Dabcyl-KTSAVLQSGFRKM-E(Edans)-NH2) and 3CLpro (WT or S301P) are prepared in 2× concentration in reaction buffer (50 mM HEPES, 0.1 mg/mL BSA, 5 mM DTT, pH 7.5). 1 μL of 50× compound 172 is added into 25 μL of 2×3CLpro and incubated at room temperature for 30 minutes on a black walled 384-well plate (ThermoFisher Scientific, USA). Then, 25 μL of 2× substrate is added to the mixture. Fluorescence (excitation/emission: 365 nm/500-550 nm) is measured by a plate reader (Glomax, Promega) after 20 minutes. The initial velocity of fluorophore release is normalized against DMSO control. GC-376 is used as a positive control throughout the experiment.

### Michaelis-Menton Inhibitory Kinetics Assay

A Michaelis-Menton inhibitory kinetic assay is conducted to compare the modes of inhibition between compound 172 and Nirmatrelvir on WT 3CLpro. Compound 172 and Nirmatrelvir are prepared in a 2-fold gradient 100× concentrations in DMSO, while substrate (Dabcyl-KTSAVLQSGFRKM-E(Edans)-NH2 SEQ ID NO:11) and WT 3CLpro are prepared in 2× concentration with reaction buffer (50 mM HEPES, 0.1 mg/mL BSA, 5 mM DTT, pH 7.5). 1 μL of 100× compound 172 or Nirmatrelvir is added to 50 μL of 2×3CLpro and incubated at room temperature for 30 minutes on a black walled 96-well plate (ThermoFisher Scientific, USA). Then, 50 μL of 2× substrate is added to each well and incubated for an additional 20 minutes, followed by measuring fluorescence (excitation/emission: 365 nm/500-550 nm) using a plate reader (Glomax, Promega). The initial velocity is calculated by dividing the fluorescent intensity by incubation time. The data is fitted into the mixed-model inhibition function to generate alpha values in GraphPad Prism. According to GraphPad Prism, when alpha value equals 1, it represents the ideal non-competitive inhibition (38). When alpha is greater than 1, the inhibitor preferentially binds to the free enzyme, and if it is very large, the model approaches competitive inhibition (38). When alpha is less than 1, the inhibitor preferentially binds to the enzyme-substrate complex, and if it is very small, the model approaches an uncompetitive model (38).

### Surface Plasmon Resonance (SPR) Spectroscopy

To verify the interaction between compound 172 and 3CLpro, a surface plasmon resonance (SPR) experiment is carried out on the Biacore T-200 machine (Cytiva, USA). First, WT or mutant 3CLpro is diluted with acetate buffer (pH 5.0) to reach a concentration of 40 μg/mL or higher and immobilized onto a Series S Sensor Chip CM5 until a response unit of 15,000 RU is achieved. Next, a running buffer containing 5% DMSO in 1×PBS is prepared, and compound 172 and GC-376 are prepared in gradient concentrations, with one concentration duplicated. DMSO is used as a negative control and background. “LMW kinetics” is selected in the Biacore software protocol wizard, and compounds are added onto a 96-well U-bottom plate based on the layout generated in the software. The reaction is started according to the software instructions. After overnight incubation, the SPR results, including the drug-protein response curve and affinity curve are generated.

### Analytical Ultracentrifugation (AUC) Assay

The sedimentation velocity experiment is carried out using a Beckman Coulter Optima analytical ultracentrifuge (Beckman Coulter, USA), equipped with an An-60 Ti rotor, at a temperature of 20° C. To examine the dimer-inhibitory effect of 172 against SARS-CoV-2 3CLpro, the 600 μg/ml freshly purified WT 3CLpro is incubated with 20 μM 172 or 0.2% DMSO for at least 2 h on ice before this assay. Then the sample is centrifuged at 40000 rpm using 12-mm standard double sector centerpieces. A total of 400 scans are taken at 4-minute intervals, featuring the data resolution at 10 μm. The detection of monomer or dimer, based on radial position and time, is achieved through absorbance measurements at 280 nm, as well as interference detection. The resulting profiles are analyzed using Sedfit software's continuous (s) distribution model.

### Golden Syrian Hamster Experiment

Golden Syrian hamsters, either male or female, aged between 4-6 months, are accommodated in a BSL-3 facility and provided with standard food pellet and water. The hamsters are grouped randomly in groups of 4 (n=4) for antiviral assessment. All experimental protocols are approved by the Animal Ethics Committee at the University of Hong Kong (CULATR), and are conducted according to the standard operating procedures of the BSL-3 animal facilities (reference code CULATR 5370-20). The treatment group hamsters received the first dose of compound 172 (dissolved in 20% SBE-BCD/0.9% saline at the concentration of 1 mg/kg), while the control group received 5% DMSO in vehicle. Both groups are administered through intraperitoneal (IP) injection three hours before infection. The experiments are not blinded. Each hamster received a nasal inoculation of 1000 PFU of Wild Type SARS-CoV-2 in 50 μl PBS while under IP anaesthesia with ketamine (200 mg/kg body weight) and xylazine (10 mg/kg body weight). Five hours after being infected with SARS-CoV-2, the hamsters received the second dose of treatment or DMSO vehicle. In the following 3 days, the hamsters are treated with a daily Q12 regimen of treatment and are monitored for signs of illness and changes in body weight. The same protocol is followed for an in vivo drug toxicity experiment, except that the virus is not inoculated. The infected hamsters are euthanized 3 days after virus inoculation for virological and histopathological examination. Their lungs and nasal turbinate are collected for viral RNA detection and plaque quantification. The tissue pathology of infected animals is examined by Haematoxylin and Eosin staining, and immunofluorescence staining, according to an established protocol (37).

### K18-hACE2 Mouse Survival Experiment

Male or female K18-hACE2 transgenic mice, aged between 6-8 weeks, are accommodated at the BSL-3 animal facility, and provided with standard food pellet and water. The use of K18-hACE2 transgenic mice has been approved by the CULATR of The University of Hong Kong under Animal Ethics Committee (reference code: 5370-20). On the day of infection, the hACE2 mice are intranasally inoculated with either 200 PFU B.1.1.7, 1 or 10,000 PFU B.1.1.529 sublineage BA.5, pre-diluted in 20 μl PBS. Mice are treated with either 50 mg/kg compound 172 in 20% SBE-BCD/0.9% saline, 200 mg/kg PF-07321332 (Nirmatrelvir) in vehicle or 5% DMSO in vehicle, by IP injection daily until sample collection or animal death. Body weight and survival of the mice is monitored on a daily basis. Mice are euthanized at designated timepoints and organ tissues are sampled for virological analyses. The mice lungs and nasal turbinate are collected for viral RNA detection and plaque quantification [25].

### Molecular Docking

Three-D structure of compound 172 is retrieved from the PubChem database (39) with ID 3700821. The high-resolution dimer structure of SARS-CoV-2 main protease (Mpro) is downloaded from the Protein Data Bank (PDB) database (40) with ID 6Y2G. Structure of Mpro bound to Pelitinib is retrieved with PDB ID 7AXM. The charge/protonation state of Mpro protein is assigned with H++ server (41). Potential ligand-binding pockets of Mpro is probed with Metapocket (42). Leadfinder v.1804 (43) is used to dock compounds against Mpro protein with extra precision mode (-xp). Intermolecular interaction plot is generated by Pymol.

### Molecular Dynamics (MD) Simulation

The best-scoring binding poses of 172-Mpro complex is used as the initial conformation and solvated in cubic water box with the TIP3P water model, extending to at least 10 Å from the protein atoms. The ff14SB force field (44) is used to describe the protein, together with general Amber force field (GAFF) (45) parameters for ligands. The simulation is carried out using the Amber17 package (46), long-range electrostatics are treated with the particle mesh Ewald method, while the van der Waals interactions are truncated at a cutoff of 10 Å. After 1 ns of equilibration, three 10 ns production runs are carried out in the NPT ensemble at 300 K. The time step is 2 fs, and snapshots are saved for analysis every 20 ps.

### Permeability of Compound 172 in Caco-2 Assay

Caco-2 cells are seeded onto 0.4 μm pore polycarbonate membranes (PC) in 96-well Corning Insert plates at a density of 3.5×104 cells/cm2.HBSS with 10.0 mM HEPES at pH 7.40±0.05 as the transport buffer. The compound is subjected to bidirectional testing in triplicate at 2.00 μM. The plate is incubated in a CO2 incubator with 5% CO₂ at 37.0° C. and saturated humidity for two hours without shaking. All samples are centrifuged at 3220 g for 10 minutes after being combined with acetonitrile containing internal standard. Using the peak area ratio of the analyte/internal standard, LC-MS/MS techniques are used to quantify the concentrations of the compound in the beginning solution, donor solution, and recipient solution. Following transportation, lucifer yellow rejection assay is applied to determine the Caco-2 cell monolayer integrity.

For data analysis, Permeability (Papp), efflux ratio and Percent recovery are calculated using the following equations:

$${P}_{app} = ( dCr / dt ) \times Vr / ( A \times {C}_{0} )$$

$$Efflux Ratio = {P}_{app} ( BA ) / {P}_{app} ( AB )$$

$$\% Solution Recovery = 100 \times [ ( {V}_{r} \times {C}_{r} ) + ( {V}_{d} \times {C}_{d} ) ] / ( {V}_{d} \times {C}_{0} )$$

Reference compounds are analysed in parallel as assay control.

### 1.24 Metabolic Stability of Compound 172 in Mouse and Human Liver Microsomes

Mouse and Human Liver Microsomes are respectively provided by RILD and coming. They are collected from CD-1 mouse or human and prepared in 100 mM potassium phosphate buffer. The reaction is started by the addition of 100 μM compound 172 working solution with or without NADPH co-factor and terminated by adding cold (4° C.) acetonitrile (ACN) containing 250 nM tolbutamide and 250 nM labetalol. Aliquots are sampled at 0, 5, 15, 30, 45, and 60 min for LC-MS/MS analysis.

### Mouse Pharmacokinetics Study

The pharmacokinetic characteristics of compound 172 after a single intraperitoneal dosage of 5 mg/kg on Balb/c mice are investigated. There are three male mice (6-10 weeks) in the experimental group and the dose volume will be determined by the animals' body weight collected on the morning of dosing day. At 0.25, 0.5, 1, 2, 4, 6, 8, and 24 hours after dosage, blood samples are obtained from saphenous vein and placed in tubes containing EDTA-K2. After 3200 g of centrifugation at 4° C. for 10 minutes, plasma samples are quickly frozen over dry ice and kept at −80° C. Identification of compound 172 in plasma is performed by LC-MS/MS analysis on DG-Triple Quad 6500 plus. Pharmacokinetic parameters are calculated by IP-Noncompartmental model 200 using Phoenix WinNonlin 8.3.5.

### Example 2: Results

### Phenotype-Based High Throughput Screening

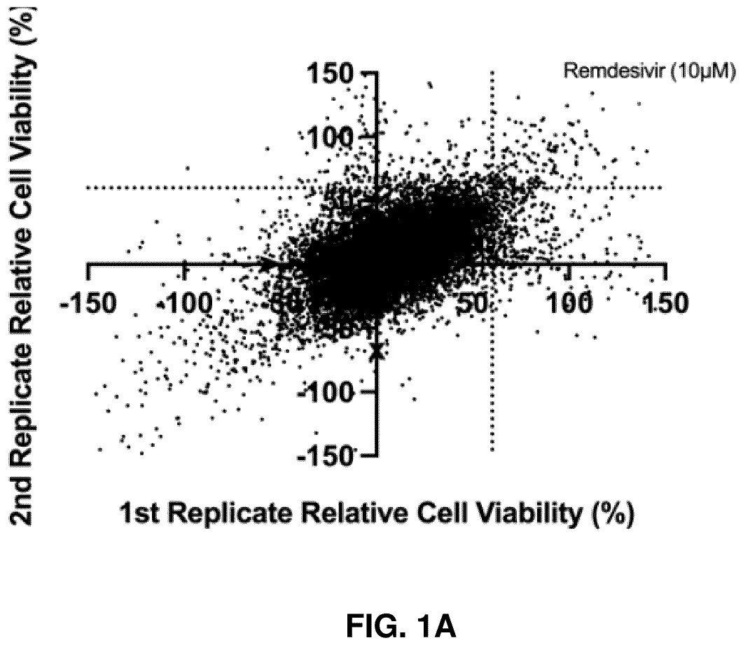

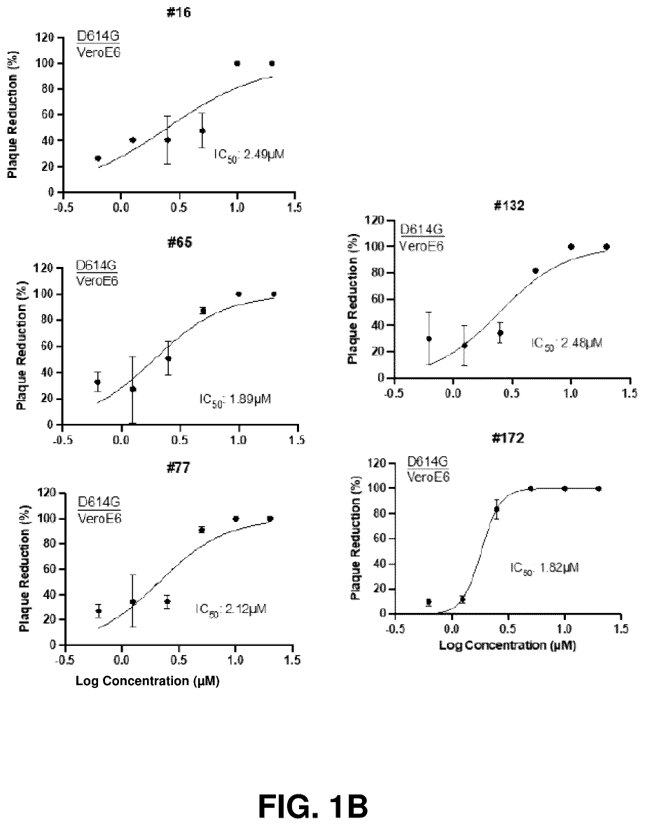

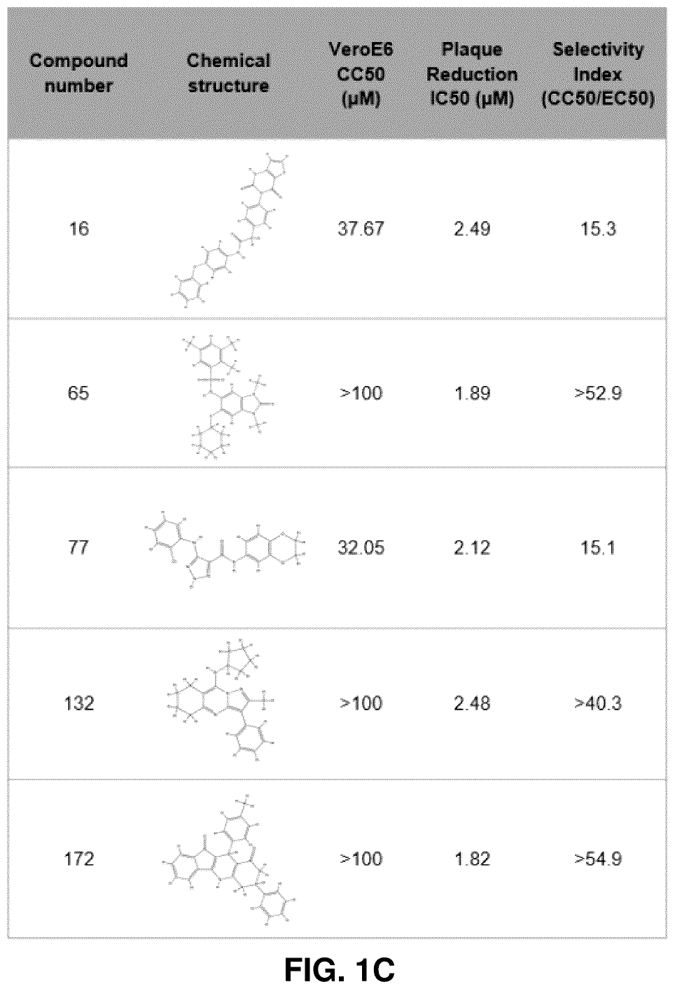

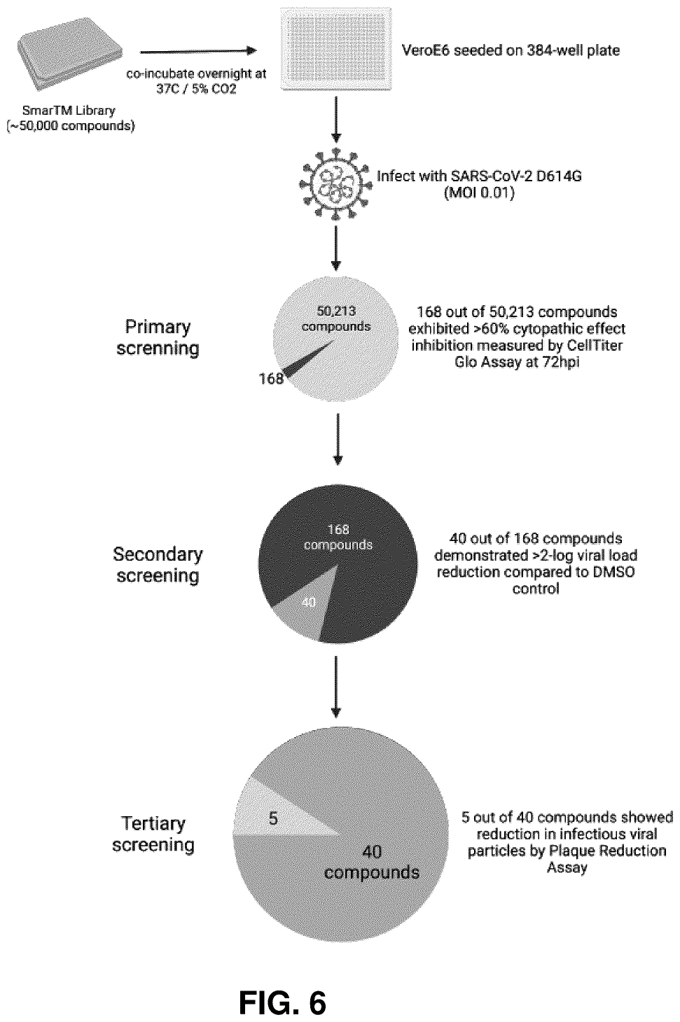

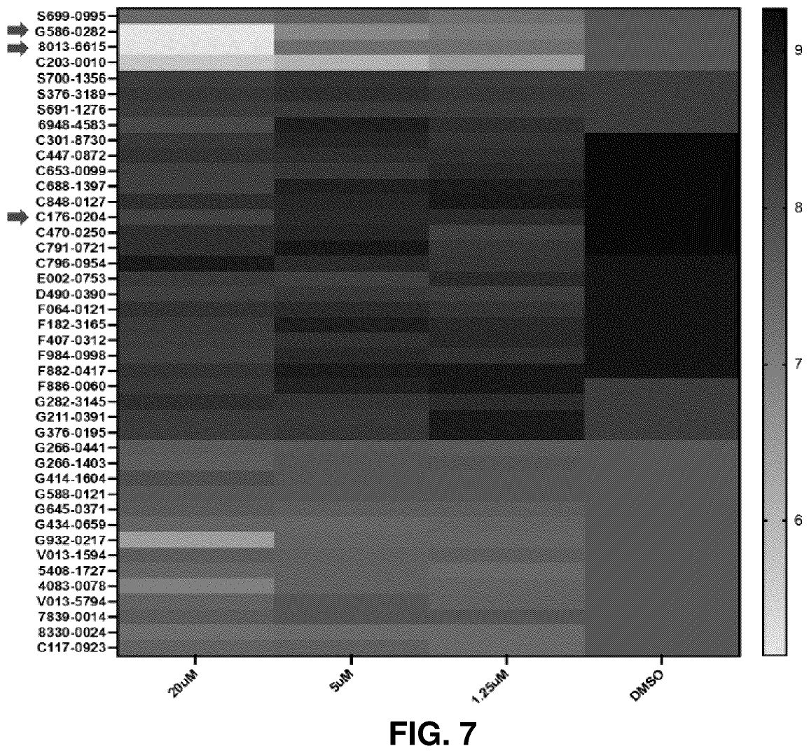

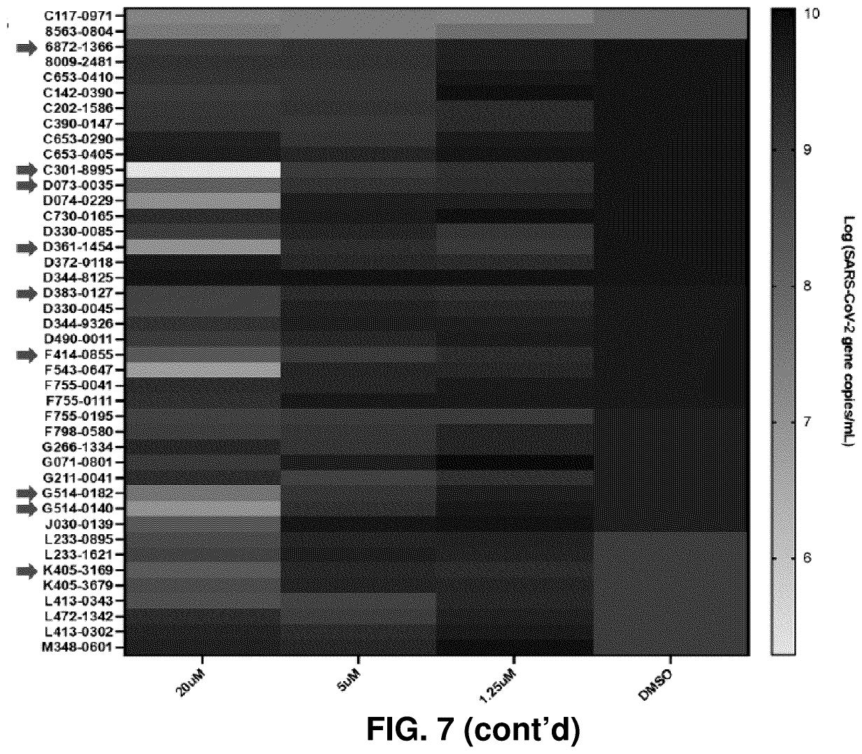

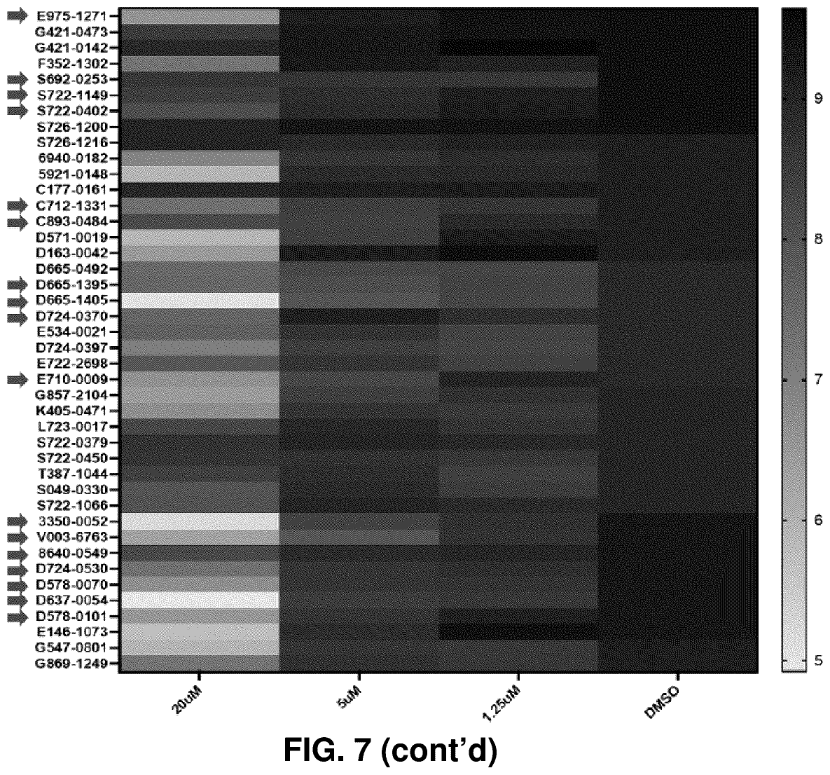

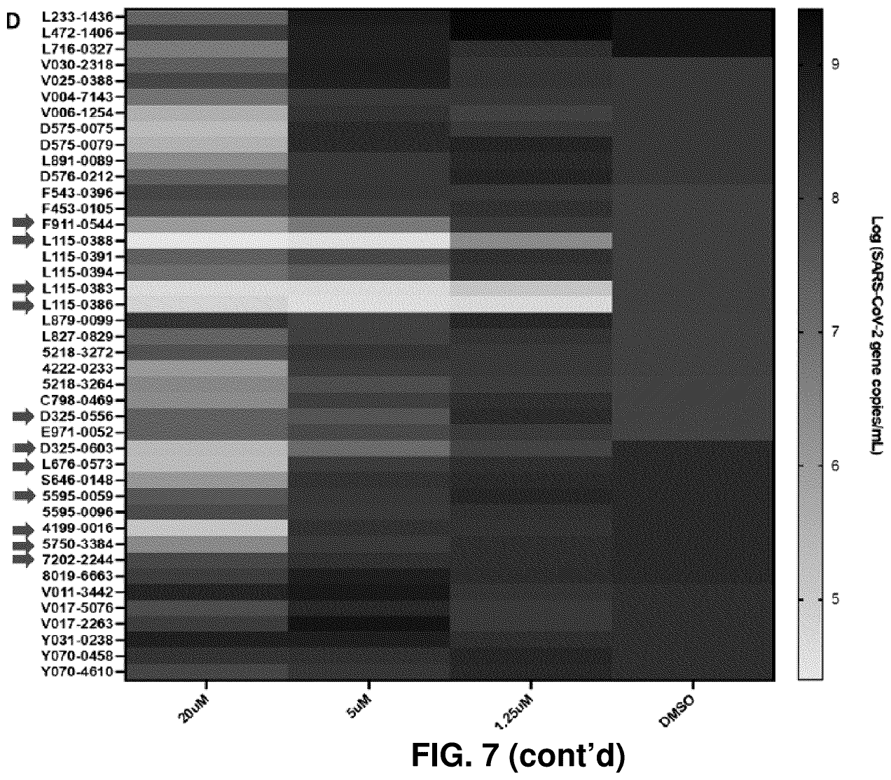

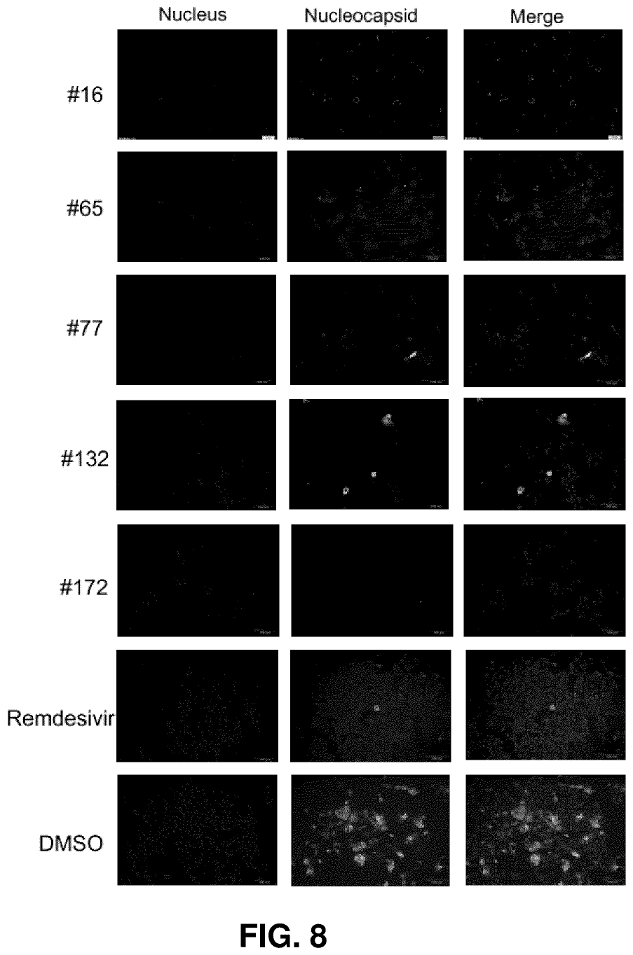

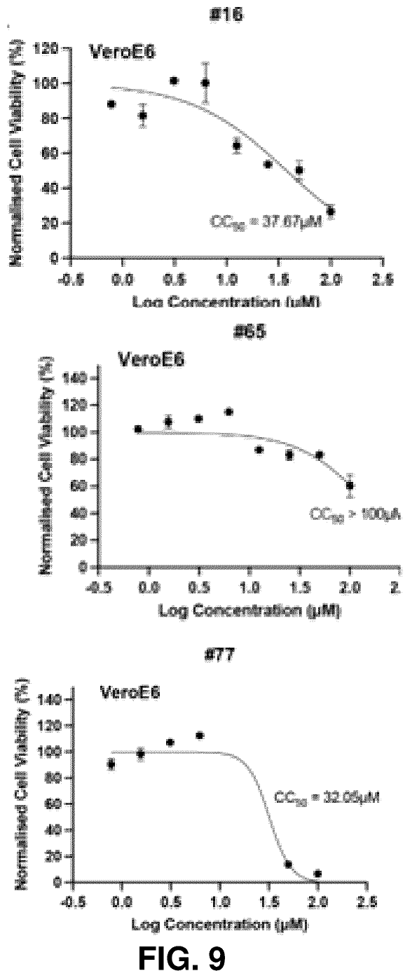

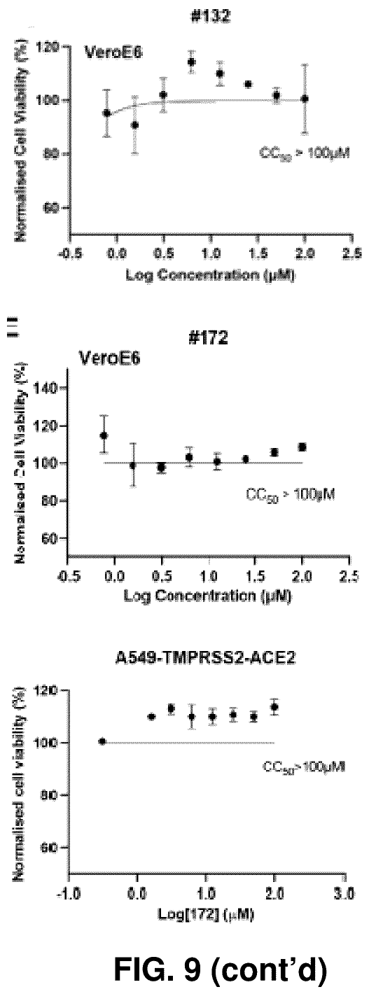

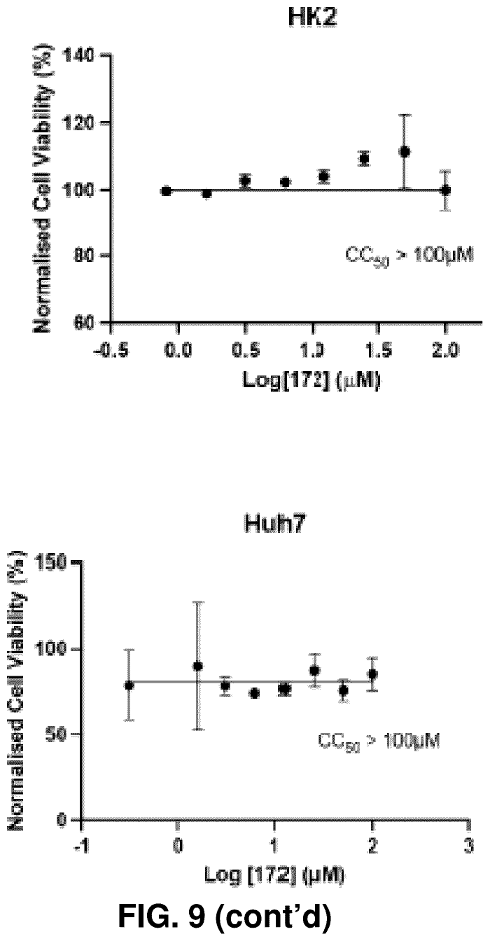

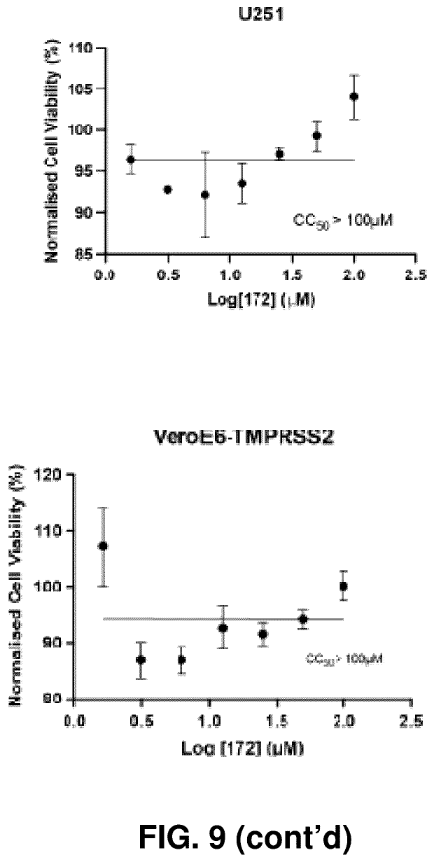

The phenotypic-based high throughput screening (HTS) is performed in three levels FIG. 6). First, primary screening is conducted by screening 50,213 synthetic compounds for their CPE inhibition (CPEi) activity against SARS-CoV-2 wildtype (WT) D614G infection in VeroE6 cells. CellTiter Glo® reagent is added into cells to measure cell viability after 72 hours-post-infection (hpi). Over 60% cell viability in both duplicates is set as a selection criterion in primary screening. As a result, 168 out of 50,213 compounds are discovered to inhibit over 60% CPE formation in VeroE6 cells at 5 μM (FIG. 1A). Afterwards, the 168 primary hits are moved onto secondary screening using human A549-TMPRSS2-ACE2 cells. Forty out of 168 compounds are found to reduce 2-log₁₀ or higher SARS-CoV-2 viral load using at least one concentration (FIG. 7). Next, 40 secondary screen hits are validated for their anti-SARS-CoV-2 activity by plaque reduction assay. Ultimately, five out of 40 compounds are confirmed to reduce anti-SARS-CoV-2 infectious particle with a 50% effective dose (IC₅₀) at lower μM levels (<5 μM, FIG. 1B) without significant cytotoxicity. These final hits, designated 16, 65, 77, 132, and 172, also reduced SARS-CoV-2 N protein expression in VeroE6-TMPRSS2 cells by immunofluorescent (IF) staining (FIG. 8). The greatest reductions in N protein expression are observed in compound 172 (FIG. 8), which is consistent with plaque reduction assay result (FIG. 1B). While the remaining four compounds exhibited moderate degree of reduction in N antigen expression (FIG. 8). MTT cytotoxicity assay is also conducted to confirm that the antiviral phenotype is not due to drug cytotoxicity (FIG. 9). The selectivity index (SI) of these five compounds is calculated as shown (FIG. 1C). Compounds 16 and 77 have lower SI (15.3 and 15.1, respectively) due to their lower CC₅₀ values (37.67 and 35.05, respectively). For compounds 65, 132 and 172, their SI are higher (>52.9, >40.3 and >54.9, respectively) which achieve CC₅₀ values higher than 100 μM.

### Prioritization of the Compounds

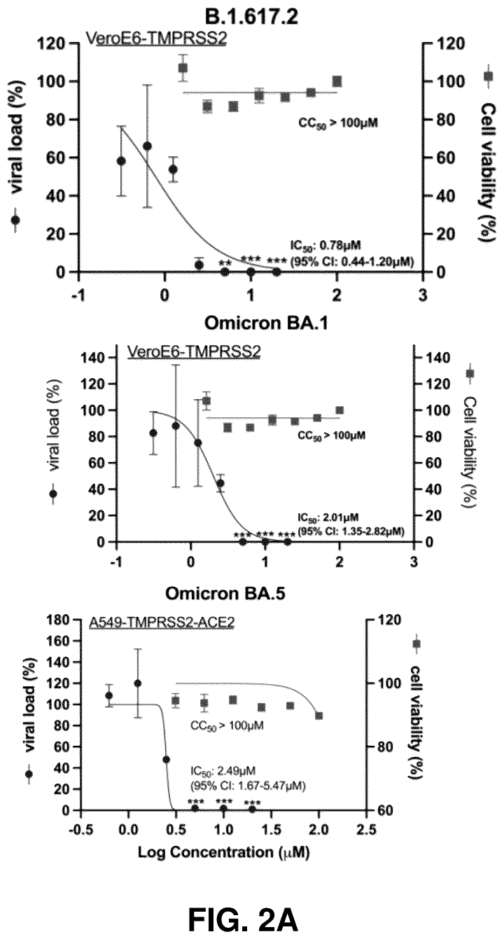

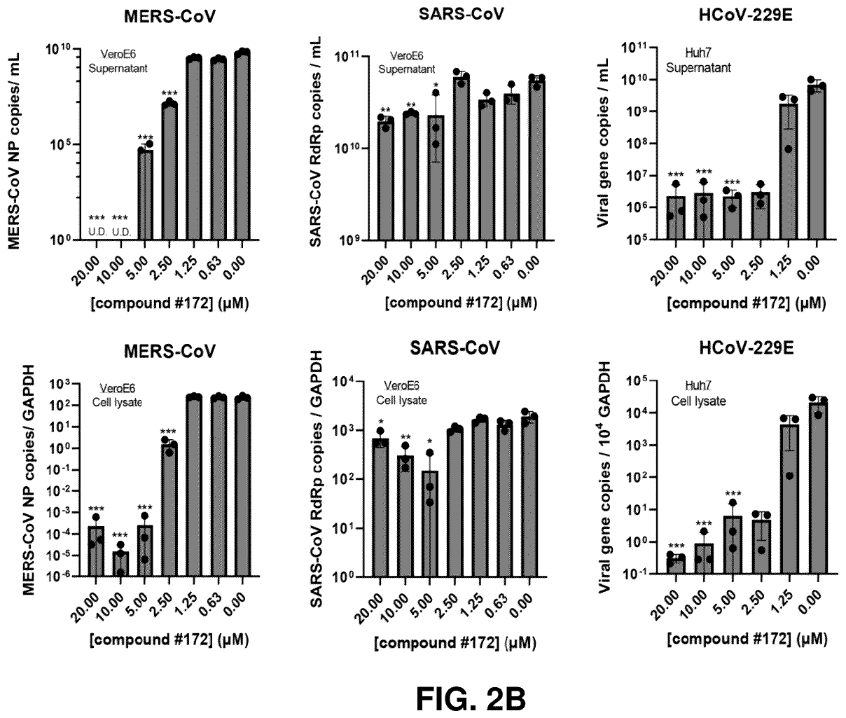

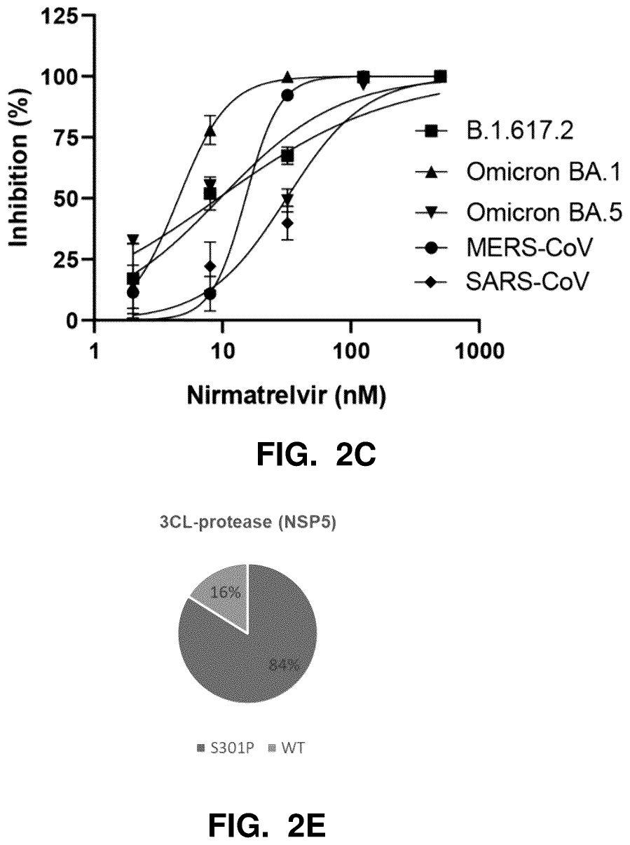

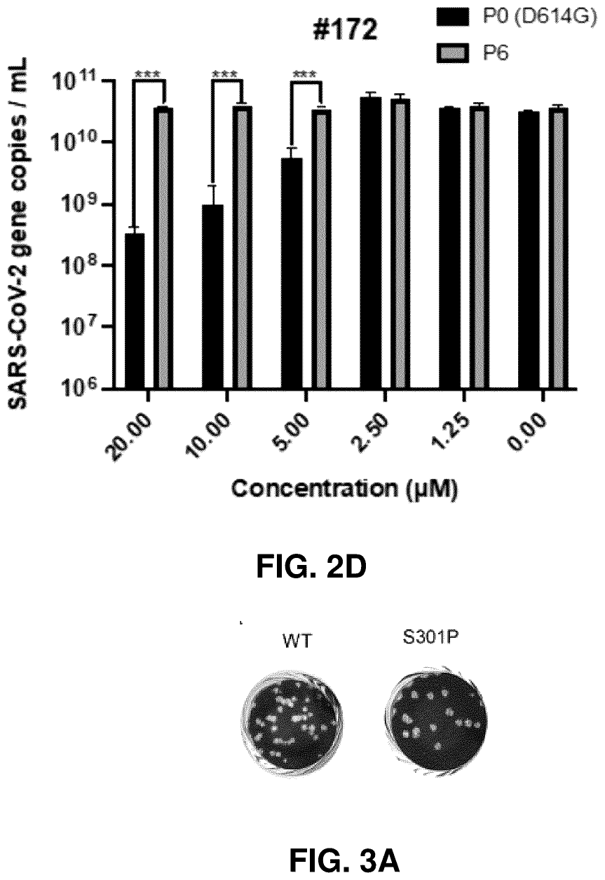

Among the five candidates, one of them (compound 172) exhibit the lowest IC50 (1.82 μM) and high CC₅₀ (>100 μM), which gives a SI >54.9 (FIG. 1C). Therefore, we decided to investigate further on its antiviral potency and mechanism. Although compound 65 has a comparable SI with 172, it is not chosen to be studied further because it demonstrated some extent of cytotoxicity in VeroE6 cells at 20 μM using plaque reduction assay, which correspond to 80% cell viability in MTT assay, yet 172-treated cells had over 90% cell viability at 100 μM and high tolerability in multiple cell lines (FIG. 9). Interestingly, compound 172 is potent against other variants of concerns (VOC) of SARS-CoV-2, such as B.1.617.2 (Delta), Omicron BA.1 and Omicron BA.5, with micromolar IC50s (FIG. 2A). Compound 172 could also inhibit the replication of MERS-CoV, SARS-CoV, and HCoV-229E in vitro and in a dose dependent manner (FIG. 2B). Notably, compound 172 reduced the MERS-CoV supernatant viral RNA level below RT-qPCR detection limit at 5 μM or above, and >5-log₁₀ reduction in the normalized expression of N gene in cell lysate (FIG. 2B). Additionally, more than 3-log 10 viral RNA reduction is observed in the supernatant of HCoV-229E infected Huh7 cells and similar results are found in the cell lysate (FIG. 2B). Lastly, compound 172 demonstrated 1-log 10 reduction of SARS-CoV viral load in the supernatant and cell lysate (FIG. 2B). As a reference control, Nirmatrelvir shows potent antiviral effects against SARS-CoV-2 variants and other human coronaviruses with a sub-nanomolar IC₅₀ values ranging from 4.5 to 32 nM (FIG. 2C). Taken together, compound 172 exhibits pan-coronavirus antiviral activity.

### Mode of Action of Compound 172

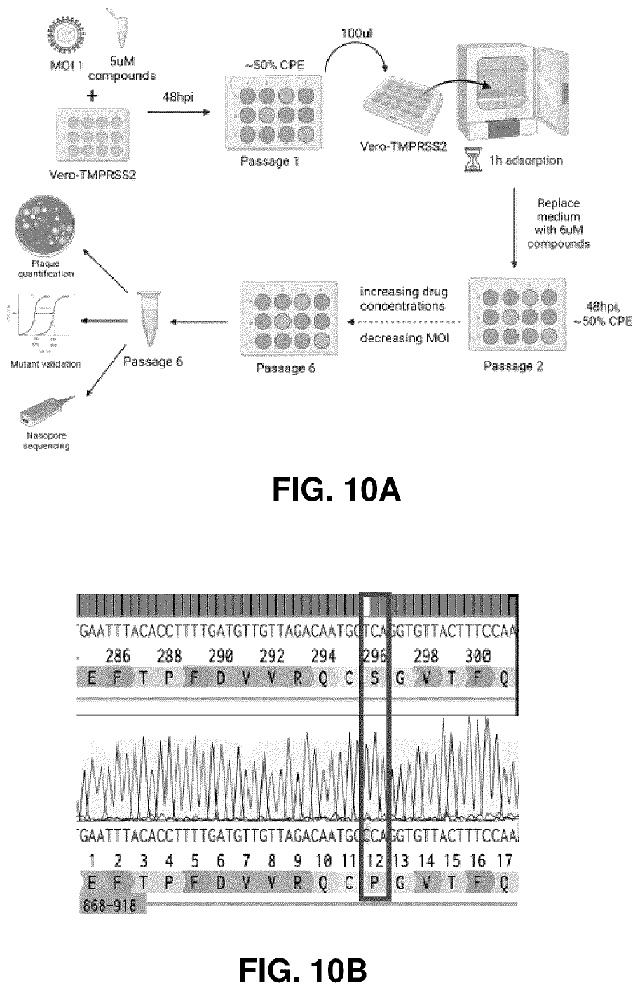

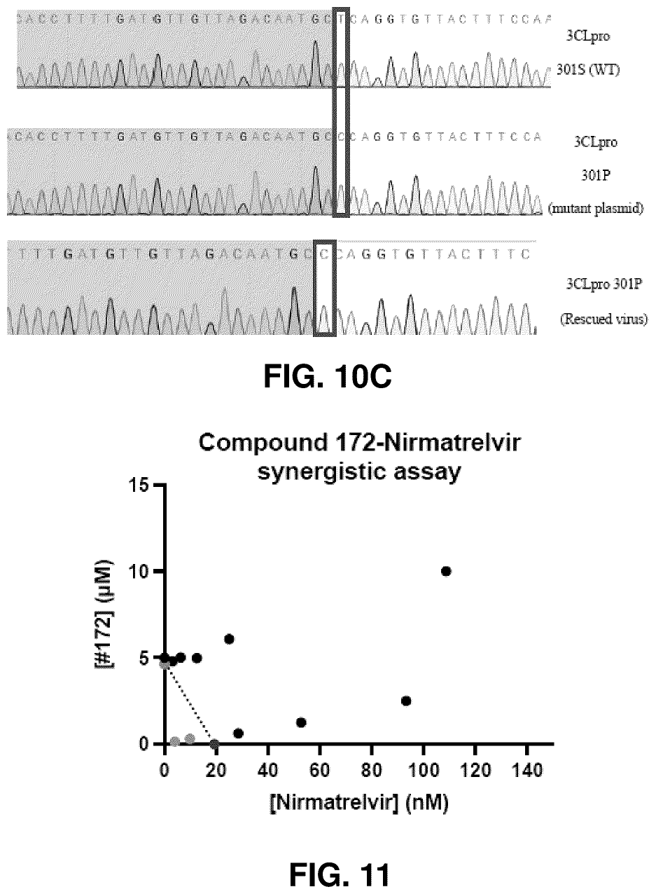

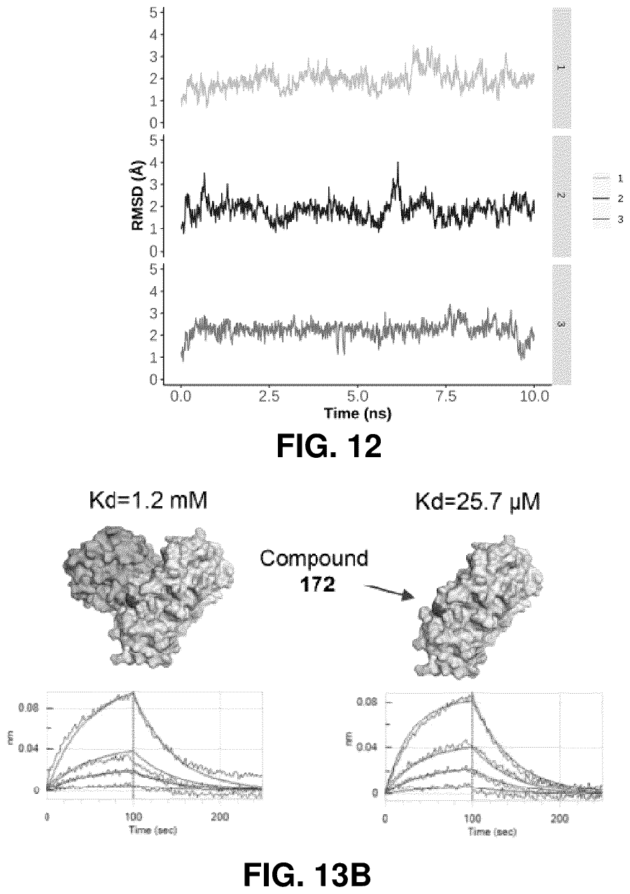

To investigate the antiviral mechanism of compound 172, an escape mutant is generated. After six rounds of passages with increasing compound 172 concentration (FIG. 10A), CPE inhibition by compound 172 is abolished at 8 μM in passage 6. Therefore, passage 6 is collected for plaque quantification and escape mutant validation (FIG. 10A). Interestingly, passage 6 is completely resistant towards compound 172 when compared to passage 0 (WT D614G), as shown by RT-qPCR analysis (FIG. 2C). Moreover, Nanopore sequencing discovered more than 80% virus population in passage 6 carried a single nonsynonymous mutation at the 10955 genome locus: a T to C nucleotide mutation, which results in the amino acid substitution of serine by proline at the 301^{st} position (S301P) of 3CLpro (FIG. 2D), when compared to that of passage 0. This single point mutation is also verified by traditional Sanger sequencing (FIG. 10B).

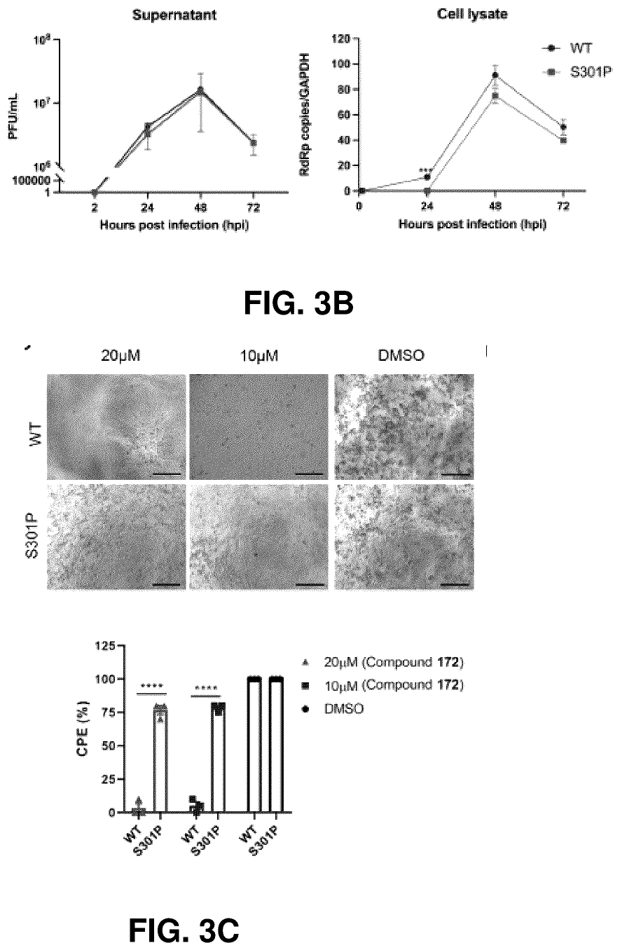

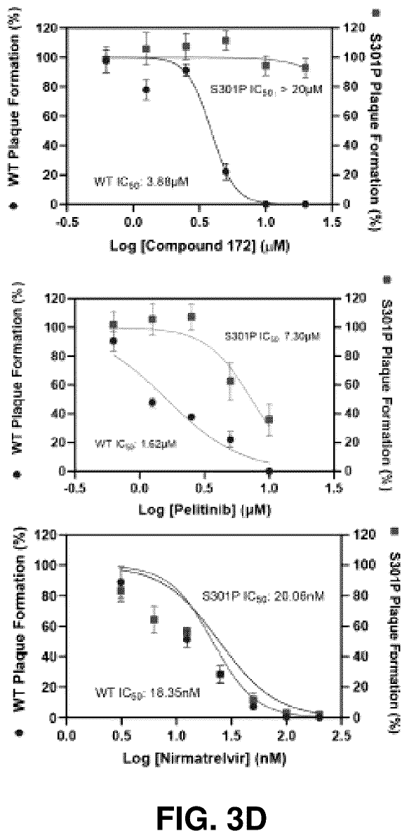

To validate the correlation between 3CLpro S301P and compound 172, a recombinant mutant virus is constructed using bacterial artificial chromosome (BAC) recombineering technology. 3CLpro S301P virus is generated and validated by traditional Sanger sequencing (FIG. 10C). The mutant virus is rescued and passaged successfully in VeroE6-TMPRSS2 cells (FIG. 3A). Replication kinetics of recombinant WT and mutant S301P virus demonstrated comparable growth rate with slight attenuation in the mutant virus (FIG. 3B). As expected, compound 172 demonstrated CPE inhibition phenotype in VeroE6-TMPRSS2 cells infected by recombinant WT virus, whereas the CPEi phenotype is abolished and cell syncytia could be observed under the infection of S301P virus (FIG. 3C). Moreover, plaque formation assay with the treatment of compound 172 resulted in an IC50 value of 3.88 μM in recombinant WT, comparable with previous screening results (FIG. 3D). However, when the cells are infected by mutant S301P virus, no plaque reduction phenotype is observed, which indicated antiviral resistance (FIG. 3D). The result also suggests that 3CLpro S301 residue is critical for the antiviral phenotype of compound 172. To further elucidate the antiviral mechanism of compound 172, the S301P virus is tested against two “reference compounds” with clarified mode of action, nirmatrelvir and Pelitinib. Nirmatrelvir is a peptidomimetic inhibitor, the active component of Paxlovid (16). Interestingly, both recombinant WT and S301P viruses are susceptible to nirmatrelvir, with similar IC₅₀ (18.35 nM and 20.06 nM) in vitro (FIG. 3D). This suggests 3CLpro S301P does not confer resistance towards nirmatrelvir and implies the antiviral mechanism of compound 172 is different from nirmatrelvir. On the other hand, Pelitinib is an epidermal growth factor receptor (EGFR) inhibitor (47). An X-ray crystallography screening study suggested that Pelitinib could interact with an allosteric site of SARS-CoV-2 3CLpro associated with S301, resulting in antiviral activity (48). Indeed, antiviral IC₅₀ of Pelitinib increased from 1.62 μM towards recombinant WT virus to 7.30 μM against the mutant virus (FIG. 3D), which verified the important role of S301 in this proposed allosteric site. Taken together, the result implied that compound 172 is a 3CLpro allosteric inhibitor associated with S301.

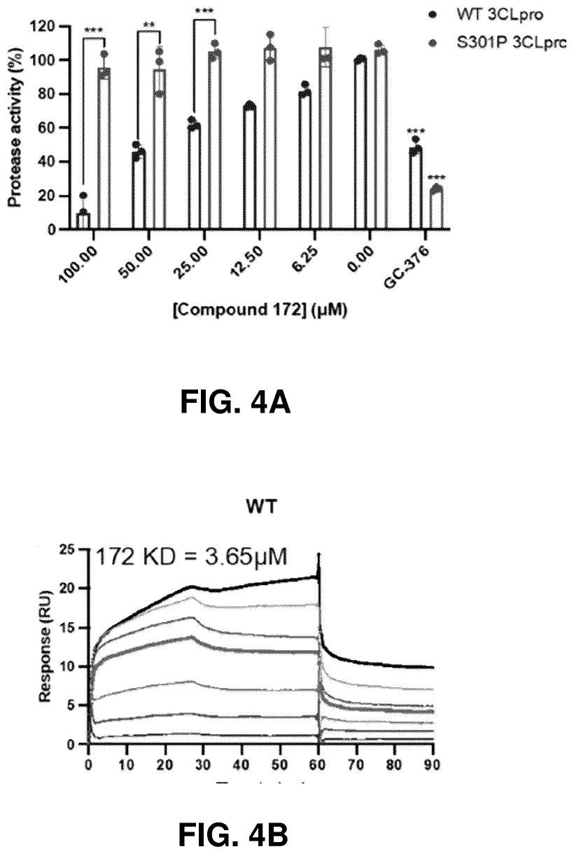

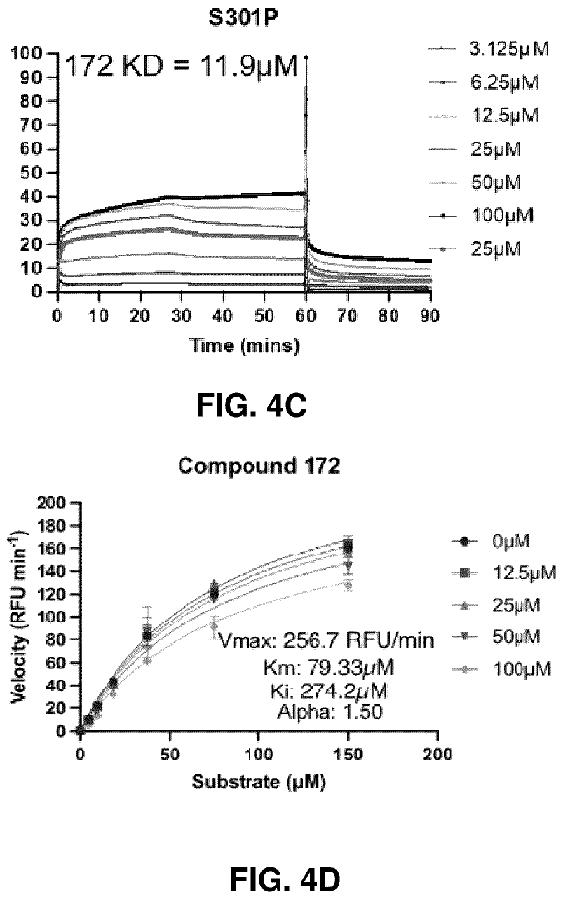

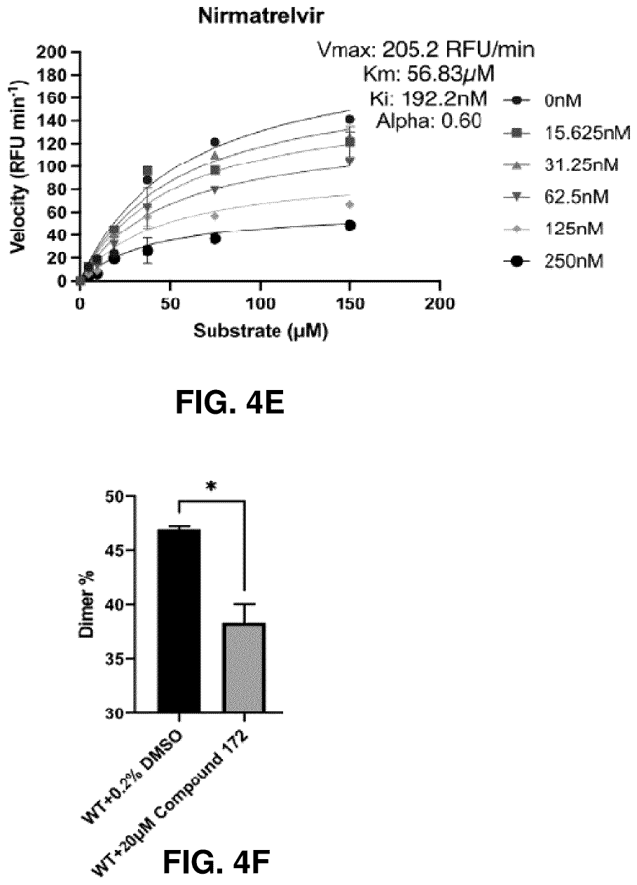

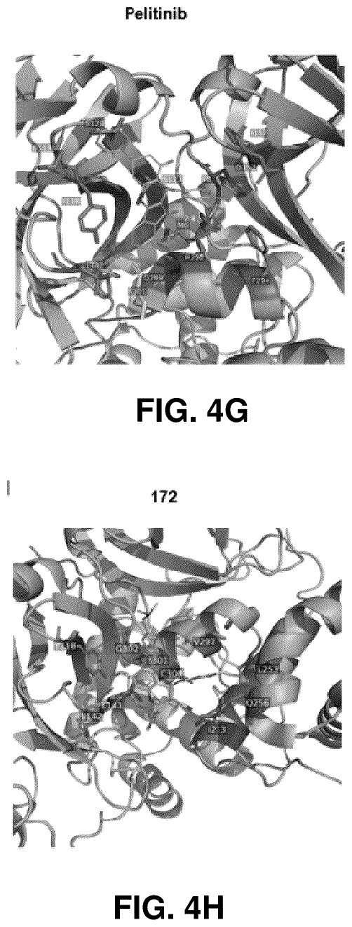

To confirm 3CLpro is the antiviral target of compound 172, WT and S301P 3CLpro are expressed and purified, for fluorescent-resonance-energy-transfer (FRET)-based protease activity assays and surface plasmon resonance (SPR) spectroscopy. Notably, compound 172 exhibited dose-dependent inhibition of WT 3CLpro cleavage activity in vitro, yet unable to inhibit S301P 3CLpro activity (FIG. 4A). Utilizing SPR spectroscopy, compound 172 is demonstrated to bind with 3CLpro with a dissociation constant (Kd) of 3.65 μM, while a higher KD is estimated for S301P protein (Kd=11.9 μM) (FIGS. 4B & 4C). Altogether, our findings confirmed that 3CLpro is the antiviral target of compound 172.

Moreover, Michaelis-Menton inhibitory kinetics is conducted to compare the mode of inhibition of compound 172 and nirmatrelvir. Notably, the inhibitory constant (Ki) for compound 172 and nirmatrelvir differ greatly: 274.2 μM and 192.2 nM (FIGS. 4D & 4E), suggesting 172 could be targeting the dimeric interface which is not revealed at native conditions. Importantly, the Alpha values for compound 172 and nirmatrelvir are 1.50 and 0.60 respectively (FIGS. 4D & 4E). This indicates compound 172 is a weak competitive inhibitor while nirmatrelvir is a non-competitive inhibitor under this experimental setup (38). The results suggest compound 172 inhibits 3CLpro by a different mode of action other than direct catalytic inhibition by nirmatrelvir.

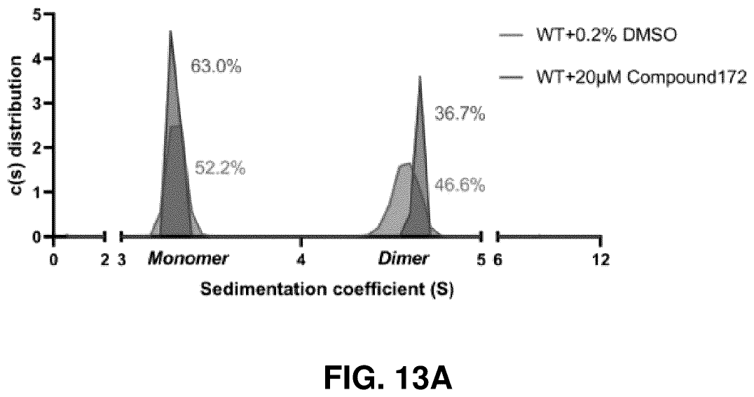

In addition, the analytical ultracentrifugation (AUC) is conducted to explore the effects of 172 on 3CLpro dimerization. Upon mixing 20 μM of compound 172 with WT 3CLpro, there is a significant reduction on 3CLpro dimerization, reducing its contribution by about 10% (FIGS. 4F and 13). Taken together, compound 172 can induce considerable effects on 3CLpro conformation by disrupting its dimerization.

On the other hand, and due to distinct MOAs between compound 172 and nirmatrelvir, it is interesting to investigate the mode of interaction between two compounds. Therefore, a drug synergism checkerboard assay is conducted. Notably, when compound 172 is at nanomolar concentration, 172-nirmatrelvir synergism could be achieved, shown by the two green points within the “isobole” (FIG. 11A). Fitting the IC50 values of nirmatrelvir with the corresponding compound 172 concentration into the Loewe's additivity (LA) equation also yielded consistent results: when compound 172 is at 312.5 nM and 156.25 nM, the LA values are smaller than 1 (0.58 and 0.24 respectively) (FIG. 4I). This indicated that 172-Nirmatrelvir drug synergism can be achieved when compound 172 is at nanomolar concentrations.

Based on the identified S301 amino acid residue responsible for compound 172 resistance (FIG. 3), molecular docking predicted that compound 172 binds to a novel allosteric site situated between the two monomers of 3CLpro, which is different from that of Pelitinib (48) (FIGS. 4G & 4H). Specifically, the allosteric site bound by Pelitinib is comprised of Y118, L141, N142, 1213, L253, Q256, V297, C300, S301 and G302 (48). However, 172's allosteric site is predicted to be surrounded by M6, F8, Y118, N119, S121, S123, L141, 1152, D153, F294, R298, Q299 and S301. Human coronavirus 3CLpro alignment analysis (for Accession number for NSP5: SARS-CoV-2 (YP_009725301.1), SARS-CoV (YP_009944370.1), MERS-CoV (YP_009047217.1), HCoV-NL63 (YP_010229075.1), HCoV-229E (NP_835349.1), HCoV-OC43 (YP_009555250.1), HCoV-HKU1 (YP_459936.1) showed that M6, Y118 and Q299 are fully conserved amino acid residues, while N119 and L141 are highly similar residues among human coronaviruses, which explains compound 172 broad spectrum activity (data not shown Furthermore, 10 ns molecular dynamics (MD) simulation showed the binding of compound 172 is thermodynamically stable, with an average root mean square deviation (RMSD) value of 2 Å (FIG. 11B).

### 2.4 In Vivo Antiviral Activity and Drug-Likeness of Compound 172

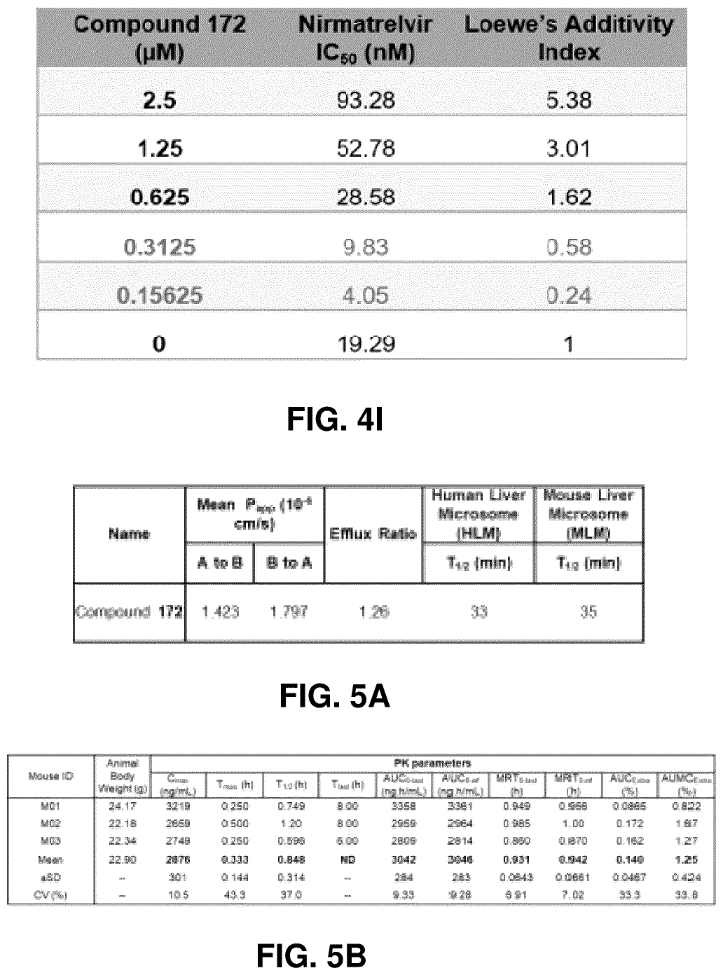

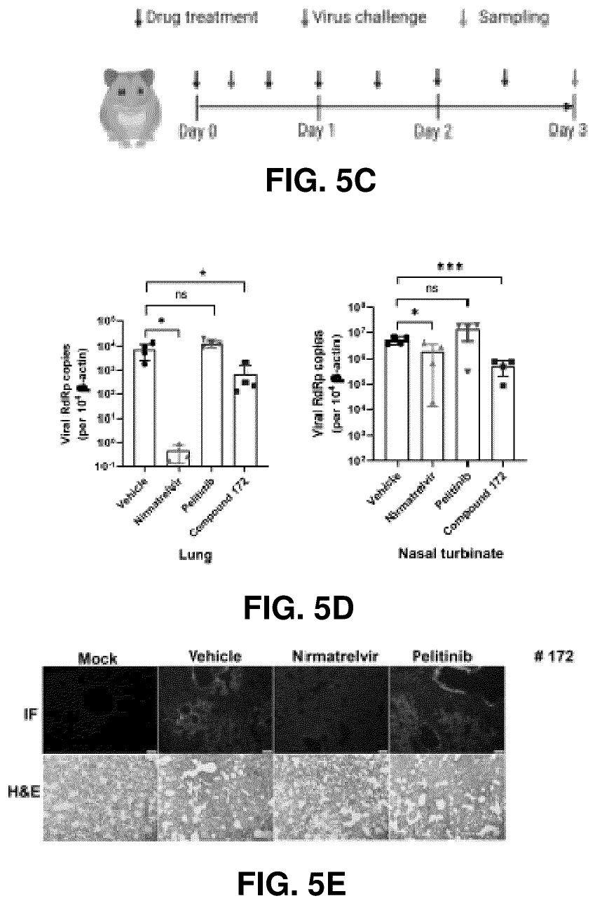

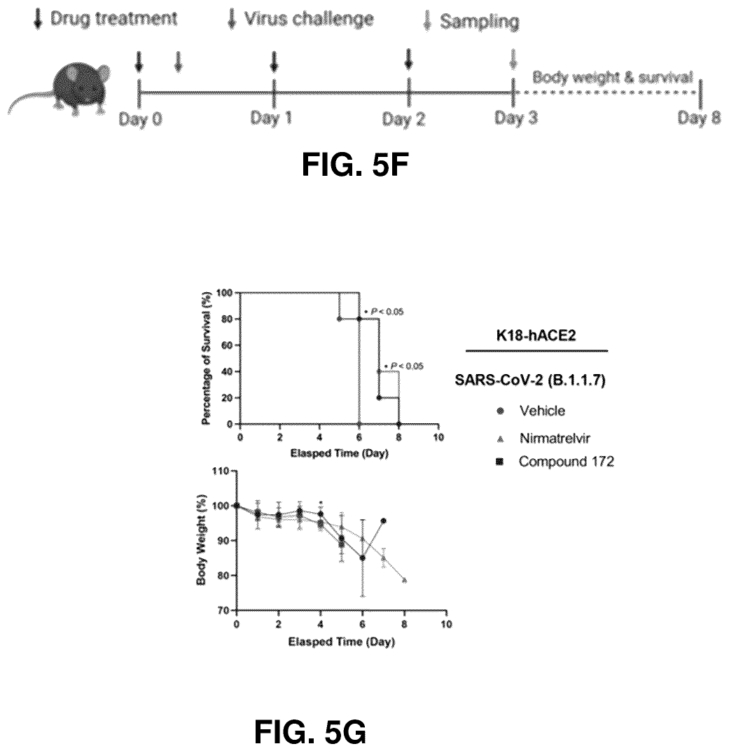

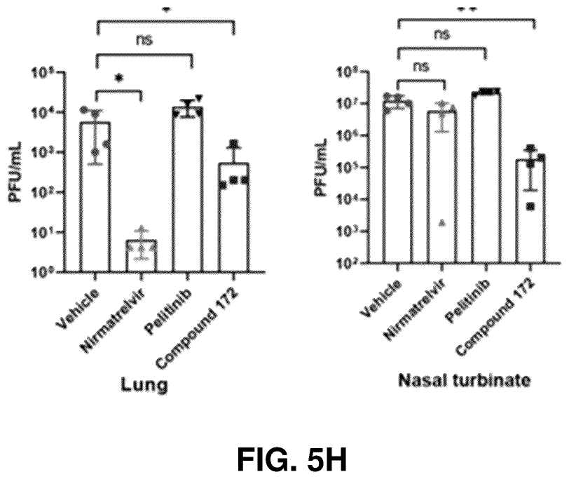

Next, the drug-likeness of compound 172 is evaluated. To explore the potential off-target effect, safety panel assays focusing on 44 selected host targets including 24 GPCRs, 8 ion channels, 7 enzymes, 3 monoamine transporters, and 2 nuclear hormone receptors are evaluated. The result suggests that compound 172 (10 μM) showed no significant off-target effects on these host proteins (see Table 1 below). Compound 172 exhibits moderate permeability and metabolic stability in cell cultures (FIG. 5A), and relatively good PK parameters in a Balb/c mouse model (FIG. 5B). The Cₘₐₓ of the compound is 6.89 μM, which is around 3.8-fold higher than its antiviral IC₅₀ of 1.82 μM. These results warrant further evaluation of compound 172's in vivo antiviral potency.

TABLE 1
In vitro safety pharmacology profiling of compound 172

| | Reference Control Data | | | | Compound 172( [symbol] ) | | | |
|---|---|---|---|---|---|---|---|---|
| Target N [symbol] | Compound [symbol] | IC [symbol] ( [symbol] M) | MaxDo [symbol] | [symbol] M [symbol] D [symbol] | Well 1 | Well 2 | Well 3 | Average |
| Alpha [symbol] | [symbol] | [symbol] | [symbol] | [symbol] | [symbol] | [symbol] | — | [symbol] |
| NET | [symbol] | [symbol] | [symbol] | [symbol] | [symbol] | [symbol] | — | [symbol] |
| Y [symbol] A | [symbol] | [symbol] | [symbol] | [symbol] | [symbol] | [symbol] | — | [symbol] |
| CC [symbol] | [symbol] | [symbol] | [symbol] | [symbol] | [symbol] | [symbol] | — | [symbol] |
| [symbol] DA | [symbol] | [symbol] | [symbol] | [symbol] | [symbol] | [symbol] | — | [symbol] |
| [symbol] | [symbol] | [symbol] | [symbol] | [symbol] | [symbol] | [symbol] | — | [symbol] |
| SHI [symbol] | [symbol] | [symbol] | [symbol] | [symbol] | [symbol] | [symbol] | — | [symbol] |
| S [symbol] T | [symbol] | [symbol] | [symbol] | [symbol] | [symbol] | [symbol] | — | [symbol] |
| DAT | [symbol] | [symbol] | [symbol] | [symbol] | [symbol] | [symbol] | — | [symbol] |
| bERG | [symbol] | [symbol] | [symbol] | [symbol] | [symbol] | [symbol] | — | [symbol] |
| [symbol] A | [symbol] | [symbol] | [symbol] | [symbol] | [symbol] | [symbol] | — | [symbol] |
| H [symbol] | [symbol] | [symbol] | [symbol] | [symbol] | [symbol] | [symbol] | — | [symbol] |
| M1 | [symbol] | [symbol] | [symbol] | [symbol] | [symbol] | [symbol] | — | [symbol] |
| M2 | [symbol] | [symbol] | [symbol] | [symbol] | [symbol] | [symbol] | — | [symbol] |
| M3 | [symbol] | [symbol] | [symbol] | [symbol] | [symbol] | [symbol] | — | [symbol] |
| A [symbol] | [symbol] | [symbol] | [symbol] | [symbol] | [symbol] | [symbol] | — | [symbol] |
| G [symbol] | [symbol] | [symbol] | [symbol] | [symbol] | [symbol] | [symbol] | — | [symbol] |
| ADORA2A | [symbol] | [symbol] | [symbol] | [symbol] | [symbol] | [symbol] | — | [symbol] |
| Alpha [symbol] A | [symbol] | [symbol] | [symbol] | [symbol] | [symbol] | [symbol] | — | [symbol] |
| GA [symbol] AA | [symbol] | [symbol] | [symbol] | [symbol] | [symbol] | [symbol] | — | [symbol] |
| D [symbol] | [symbol] | [symbol] | [symbol] | [symbol] | [symbol] | [symbol] | — | [symbol] |
| D [symbol] | [symbol] | [symbol] | [symbol] | [symbol] | [symbol] | [symbol] | — | [symbol] |
| [symbol] delta | [symbol] | [symbol] | [symbol] | [symbol] | [symbol] | [symbol] | — | [symbol] |
| [symbol] kappa | [symbol] | [symbol] | [symbol] | [symbol] | [symbol] | [symbol] | — | [symbol] |
| [symbol] | [symbol] | [symbol] | [symbol] | [symbol] | [symbol] | [symbol] | — | [symbol] |
| [symbol] T [symbol] | [symbol] | [symbol] | [symbol] | [symbol] | [symbol] | [symbol] | — | [symbol] |
| [symbol] T [symbol] | [symbol] | [symbol] | [symbol] | [symbol] | [symbol] | [symbol] | — | [symbol] |
| B [symbol] | [symbol] | [symbol] | [symbol] | [symbol] | [symbol] | [symbol] | — | [symbol] |
| Beta1 | [symbol] | [symbol] | [symbol] | [symbol] | [symbol] | [symbol] | — | [symbol] |
| Beta2 | [symbol] | [symbol] | [symbol] | [symbol] | [symbol] | [symbol] | — | [symbol] |
| [symbol] CH [symbol] | [symbol] | [symbol] | [symbol] | [symbol] | [symbol] | [symbol] | — | [symbol] |
| [symbol] T [symbol] | [symbol] | [symbol] | [symbol] | [symbol] | [symbol] | [symbol] | — | [symbol] |
| C [symbol] 1 | [symbol] | [symbol] | [symbol] | [symbol] | [symbol] | [symbol] | — | [symbol] |
| C [symbol] 2 | [symbol] | [symbol] | [symbol] | [symbol] | [symbol] | [symbol] | — | [symbol] |
| N [symbol] | [symbol] | [symbol] | [symbol] | [symbol] | [symbol] | [symbol] | — | [symbol] |
| [symbol] | [symbol] | [symbol] | [symbol] | [symbol] | [symbol] | [symbol] | — | [symbol] |
| ACH [symbol] | [symbol] | [symbol] | [symbol] | [symbol] | [symbol] | [symbol] | [symbol] | [symbol] |
| COX1 | [symbol] | [symbol] | [symbol] | [symbol] | [symbol] | [symbol] | [symbol] | [symbol] |
| COX2 | [symbol] | [symbol] | [symbol] | [symbol] | [symbol] | [symbol] | [symbol] | [symbol] |
| L [symbol] K | [symbol] | [symbol] | [symbol] | [symbol] | [symbol] | [symbol] | [symbol] | [symbol] |
| MAO [symbol] | [symbol] | [symbol] | [symbol] | [symbol] | [symbol] | [symbol] | [symbol] | [symbol] |
| PD [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| [symbol] A [symbol] | [symbol] | [symbol] | [symbol] | [symbol] | [symbol] | [symbol] | [symbol] | [symbol] |

[symbol]

indicates data missing or illegible when filed

To evaluate the prelincial potential of compound 172, it is tested in both golden Syrian hamster and K18-hACE20 mouse models. To investigate the antiviral activity of compound 172 in Golden Syrian hamster, the hamsters (n=4) are infected with 1000 PFU WT SARS-CoV-2 virus and treated with 1 mg/kg compound 172 (treatment) or Vehicle (control) or Nirmatrelvir (200 mg/kg) by intrapretoneal (IP) injection or Pelitinib (oral 10 mg/kg) at 12-hour intervals (FIG. 5C). Notably, the lung and nasal turbinate (NT) of the hamsters in the 172-treated group exhibited a 1-log 10 reduction when compared to the control group (FIG. 5D). The protection by Nirmatrelvir is extraordinarily potent in animal lungs with >4-log 10 reduction, whereas that from Pelitinib is marginal (FIG. 5D). Histopathological and IF staining of the lungs of the 172-treated group also showed a lower degree of lymphocyte infiltration and reduced SARS-COV-2 N antigen expression when compared to the control group (FIG. 5E).

On the other hand, compound 172 is also tested in the K18-hACE2 transgenic mice model. For the survival study, K18-hACE2 transgenic mice (n=5) are challenged with 200 PFU SARS-COV-2 Alpha variant (B.1.1.7) and treated with either 50 mg/kg compound 172 (treatment), 5% DMSO in vehicle (vehicle control), or 200 mg/kg Nirmatrelvir (positive control), by IP injection once daily, until death or reaching the humane endpoint (FIG. 5F). A higher drug dosage of 172 is administered due to the high susceptibility of K18-hACE2 mice to Alpha SARS-CoV-2 infection. IP injection is utilized to enhance the systemic dissemination of both compound 172 and Nirmatrelvir. In the vehicle control group, one mouse died on day 5, and all remaining mice died on day 6 (FIG. 5G). However, in the 172-treated groups, a delayed time of death is observed as one mouse died on day 6, and all remaining mice died on day 7 and/or 8 (FIG. 5G). The same delay in death is observed in the Nirmatrelvir group, where one mouse died on day 6, and all remaining mice died on day 8 (FIG. 5G). The mice's body weight is monitored daily, and a significant benefit in weight percentage is observed between the 172 and the vehicle control group on day 4 (FIG. 5G). For the viral load study, K18-hACE2 transgenic mice are infected with 10,000 PFU Omicron BA.5 variant. In line with the finding in hamsters, compound 172 can reduce live virus titer in both upper and lower respiratory tract of infected mice (FIG. 5H). Taken together, these findings suggest that compound 172 is effective against SARS-CoV-2 infection in vivo.

Additionally, the drug-likeness of compound 172 is also investigated (FIG. 5H). Using an ADME calculator, compound 172 structure is evaluated by a number of parameters derived from the Lipinski's Rule of Five (49, 50). These parameters include number of H donors/acceptors, molecular weight, octanol-water partition coefficient (LogP), molar refractivity and topological polar surface area (TPSA) (FIG. 5H) (49, 50). Apparently, compound 172 fulfilled most of the criteria except LogP, which suggests its clinical potential for further structural optimization (FIG. 5H). In summary, compound 172 exhibited anti-SARS-CoV-2 activity in vitro and in vivo.

### Example 3: Discussion

The SARS-CoV-2 3CLpro is also known as Main Protease, which plays a crucial role in processing the viral polyprotein during early replication by cleaving at specific sequences between NSP5-16 (15, 51, 52). The 3CLpro is conserved among coronaviruses and functions as a dimer with three domains (15). Domains I (residues 10-99) and II (residues 100-182) are homologues of the 3C protease of Picornaviruses, due to similar secondary structures (15, 53). The catalytic dyads (C145 and H41) are situated within Domain I and II (15). However, domain III (residues 198-303) is uniquely found in coronavirus and is involved in the dimerization of the protease (15, 54-57). Since the 172-resistant S301P mutation in the 3CLpro is within domain III, the compound is likely to interfere with 3CLpro dimerization. Our AUC assay directly demonstrated the compound 172 can disrupt the 3CLpro dimerization, inducing considerable changes on 3CLpro secondary structures (FIG. 4F). Furthermore, the 3CLpro S301P mutant only conferred resistance towards compound 172 but not Nirmatrelvir (FIGS. 3D & 3G). These results suggested that compound 172 inhibits the 3CLpro via a distinct mechanism from Nirmatrelvir.

The association of S301 with an allosteric site on 3CLpro has been reported in a previous x-ray crystallography screening (48). One of the screened compounds, Pelitinib, is predicted to interact with the S301 and bind to an allosteric site situated between the 3CLpro monomers (48). Notably, Pelitinib exhibited a dose-dependent inhibition of WT virus replication but not the mutant virus. This confirms that the S301 residue is essential for Pelitinib's antiviral activity. Comparing with 172, however, Pelitinib showed considerable high cytotoxicity and off-target effects as a repurposed anti-cancer treatment. Molecular docking predicted that compound 172 binds to an allosteric site different from Pelitinib (FIGS. 4G & 4H). The C-terminal sequence of 3CLpro forms an alpha helix (FIG. 4H). Since proline is known to destabilize alpha helix structure (58), S301P could alter the secondary structure of the C-terminal sequences, thus disrupting the binding pocket of 172. Several previous studies have reported that substrate-binding is a crucial factor for 3CLpro dimerization (59-62), inducing the opposite effect of compound 172. Therefore, there is a negative feedback relationship between substrate-binding and 172-binding, which explains the low competitiveness of compound 172. Taken together, these findings indicate compound 172 inhibits 3CLpro via disrupting 3CLpro dimerization, by binding to a novel allosteric site associated with S301. Three out of five fully conserved and two out of five highly similar amino acids are found within the 172 binding pocket (data not shown), which explains the pan-coronavirus activity of the compound. Lastly, the monomeric form of 3CLpro is expressed and purified for affinity assay. Compound 172 is discovered to have 46-fold higher affinity towards the monomer, further validate 172 binding to the dimeric interface of 3CLpro (FIG. 13B).

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Those skilled in the art will recognize, or be able to ascertain, using no more than routine experimentation, many equivalents to the specific embodiments of the method and compositions described herein. Such equivalents are intended to be encompassed by the following claims.

### REFERENCES

1. Hu Y, Lewandowski E M, Tan H, Zhang X, Morgan R T, Zhang X, et al. Naturally Occurring Mutations of SARS-CoV-2 Main Protease Confer Drug Resistance to Nirmatrelvir. ACS Central Science. 2023; 9(8):1658-69.
2. Stevens L J, Pruijssers A J, Lee H W, Gordon C J, Tchesnokov E P, Gribble J, et al. Mutations in the SARS-CoV-2 RNA-dependent RNA polymerase confer resistance to remdesivir by distinct mechanisms. Sci Transl Med. 2022; 14(656):eabo0718.
3. Iketani S, Mohri H, Culbertson B, Hong S J, Duan Y, Luck M I, et al. Multiple pathways for SARS-CoV-2 resistance to nirmatrelvir. Nature. 2023; 613(7944):558-64.
4. Kiso M, Furusawa Y, Uraki R, Imai M, Yamayoshi S, Kawaoka Y. In vitro and in vivo characterization of SARS-CoV-2 strains resistant to nirmatrelvir. Nature Communications. 2023; 14(1):3952.
5. Merches K, Breunig L, Fender J, Brand T, Batz V, Idel S, et al. The potential of remdesivir to affect function, metabolism and proliferation of cardiac and kidney cells in vitro. Arch Toxicol. 2022; 96(8):2341-60.
6. Zhou S, Hill C S, Sarkar S, Tse L V, Woodburn B M D, Schinazi R F, et al. beta-d-N4-hydroxycytidine Inhibits SARS-CoV-2 Through Lethal Mutagenesis But Is Also Mutagenic To Mammalian Cells. J Infect Dis. 2021; 224(3):415-9.
7. Peiris J S, Lai S T, Poon L L, Guan Y, Yam L Y, Lim W, et al. Coronavirus as a possible cause of severe acute respiratory syndrome. Lancet. 2003; 361(9366):1319-25.
8. Zaki A M, van Boheemen S, Bestebroer T M, Osterhaus A D, Fouchier R A. Isolation of a novel coronavirus from a man with pneumonia in Saudi Arabia. The New England journal of medicine. 2012; 367(19):1814-20.
9. Zhou P, Yang X L, Wang X G, Hu B, Zhang L, Zhang W, et al. A pneumonia outbreak associated with a new coronavirus of probable bat origin. Nature. 2020; 579(7798):270-3.
10. Chan J F, Yuan S, Kok K H, To K K, Chu H, Yang J, et al. A familial cluster of pneumonia associated with the 2019 novel coronavirus indicating person-to-person transmission: a study of a family cluster. Lancet. 2020; 395(10223):514-23.
11. Guan W J, Ni Z Y, Hu Y, Liang W H, Ou C Q, He J X, et al. Clinical Characteristics of Coronavirus Disease 2019 in China. N Engl J Med. 2020; 382(18):1708-20.
12. Chen N, Zhou M, Dong X, Qu J, Gong F, Han Y, et al. Epidemiological and clinical characteristics of 99 cases of 2019 novel coronavirus pneumonia in Wuhan, China: a descriptive study. Lancet. 2020; 395(10223):507-13.
13. Huang C, Wang Y, Li X, Ren L, Zhao J, Hu Y, et al. Clinical features of patients infected with 2019 novel coronavirus in Wuhan, China. Lancet. 2020; 395(10223):497-506.
14. Menni C, Valdes A M, Polidori L, Antonelli M, Penamakuri S, Nogal A, et al. Symptom prevalence, duration, and risk of hospital admission in individuals infected with SARS-CoV-2 during periods of omicron and delta variant dominance: a prospective observational study from the ZOE COVID Study. Lancet. 2022; 399(10335):1618-24.
15. Zhang L, Lin D, Sun X, Curth U, Drosten C, Sauerhering L, et al. Crystal structure of SARS-CoV-2 main protease provides a basis for design of improved & #x3b1;-ketoamide inhibitors. Science. 2020; 368(6489):409-12.
16. Owen D R, Allerton C M N, Anderson A S, Aschenbrenner L, Avery M, Berritt S, et al. An oral SARS-CoV-2 M(pro) inhibitor clinical candidate for the treatment of COVID-19. Science. 2021; 374(6575):1586-93.
17. Gao Y, Yan L, Huang Y, Liu F, Zhao Y, Cao L, et al. Structure of the RNA-dependent RNA polymerase from COVID-19 virus. Science. 2020; 368(6492):779-82.
18. Liu L, Iketani S, Guo Y, Chan J F W, Wang M, Liu L, et al. Striking antibody evasion manifested by the Omicron variant of SARS-CoV-2. Nature. 2022; 602(7898):676-81.
19. Gordon C J, Tchesnokov E P, Woolner E, Perry J K, Feng J Y, Porter D P, et al. Remdesivir is a direct-acting antiviral that inhibits RNA-dependent RNA polymerase from severe acute respiratory syndrome coronavirus 2 with high potency. J Biol Chem. 2020; 295(20):6785-97.
20. Gordon C J, Tchesnokov E P, Schinazi R F, Götte M. Molnupiravir promotes SARS-CoV-2 mutagenesis via the RNA template. J Biol Chem. 2021; 297(1):100770.
21. Mukae H, Yotsuyanagi H, Ohmagari N, Doi Y, Sakaguchi H, Sonoyama T, et al. Efficacy and Safety of Ensitrelvir in Patients With Mild-to-Moderate Coronavirus Disease 2019: The Phase 2b Part of a Randomized, Placebo-Controlled, Phase 2/3 Study. Clinical Infectious Diseases. 2022; 76(8):1403-11.
22. Beigel J H, Tomashek K M, Dodd L E, Mehta A K, Zingman B S, Kalil A C, et al. Remdesivir for the Treatment of Covid-19—Final Report. N Engl J Med. 2020; 383(19):1813-26.
23. Jayk Bernal A, Gomes da Silva M M, Musungaie D B, Kovalchuk E, Gonzalez A, Delos Reyes V, et al. Molnupiravir for Oral Treatment of Covid-19 in Nonhospitalized Patients. N Engl J Med. 2022; 386(6):509-20.
24. Xie Y, Yin W, Zhang Y, Shang W, Wang Z, Luan X, et al. Design and development of an oral remdesivir derivative VV116 against SARS-CoV-2. Cell Research. 2021; 31(11):1212-4.
25. Zhang J L, Li Y H, Wang L L, Liu H Q, Lu S Y, Liu Y, et al. Azvudine is a thymus-homing anti-SARS-CoV-2 drug effective in treating COVID-19 patients. Signal Transduct Target Ther. 2021; 6(1):414.
26. Unoh Y, Uehara S, Nakahara K, Nobori H, Yamatsu Y, Yamamoto S, et al. Discovery of S-217622, a Noncovalent Oral SARS-CoV-2 3CL Protease Inhibitor Clinical Candidate for Treating COVID-19. J Med Chem. 2022; 65(9):6499-512.
27. Zhan Y, Lin Z, Liang J, Sun R, Li Y, Lin B, et al. Leritrelvir for the treatment of mild or moderate COVID-19 without co-administered ritonavir: a multicentre randomised, double-blind, placebo-controlled phase 3 trial. EClinicalMedicine. 2024; 67:102359.
28. Wang F, Xiao W, Tang Y, Cao M, Shu D, Asakawa T, et al. Efficacy and safety of SIM0417 (SSD8432) plus ritonavir for COVID-19 treatment: a randomised, double-blind, placebo-controlled, phase 1b trial. Lancet Reg Health West Pac. 2023; 38:100835.
29. Choi H, Kim J Y, Chang Y T, Nam H G. Forward chemical genetic screening. Methods Mol Biol. 2014; 1062:393-404.
30. Cong F, Cheung A K, Huang S M. Chemical genetics-based target identification in drug discovery. Annu Rev Pharmacol Toxicol. 2012; 52:57-78.
31. Zhao Y, Fang C, Zhang Q, Zhang R, Zhao X, Duan Y, et al. Crystal structure of SARS-CoV-2 main protease in complex with protease inhibitor PF-07321332. Protein Cell. 2022; 13(9):689-93.
32. Yuan S, Yin X, Meng X, Chan J F, Ye Z W, Riva L, et al. Clofazimine broadly inhibits coronaviruses including SARS-CoV-2. Nature. 2021; 593(7859):418-23.
33. Yuan S, Chu H, Huang J, Zhao X, Ye Z W, Lai P M, et al. Viruses harness YxxØ motif to interact with host AP2M1 for replication: A vulnerable broad-spectrum antiviral target. Sci Adv. 2020; 6(35):eaba7910.
34. Lewandowski K, Xu Y, Pullan S T, Lumley S F, Foster D, Sanderson N, et al. Metagenomic Nanopore Sequencing of Influenza Virus Direct from Clinical Respiratory Samples. J Clin Microbiol. 2019; 58(1).
35. J. Q. Artic Network-nCoV 2019 sequencing protocol 2020 [Available from: https://artic.network/ncov-2019.
36. Liang R, Ye Z W, Ong C P, Qin Z, Xie Y, Fan Y, et al. The spike receptor-binding motif G496S substitution determines the replication fitness of SARS-CoV-2 Omicron sublineage. Emerg Microbes Infect. 2022; 11(1):2093-101.
37. Yuan S, Chu H, Chan J F, Ye Z W, Wen L, Yan B, et al. SREBP-dependent lipidomic reprogramming as a broad-spectrum antiviral target. Nat Commun. 2019; 10(1):120.
38. Copeland R A. Evaluation of enzyme inhibitors in drug discovery: a guide for medicinal chemists and pharmacologists: John Wiley & Sons; 2013.
39. Kim S, Chen J, Cheng T, Gindulyte A, He J, He S, et al. PubChem 2023 update. Nucleic Acids Res. 2023; 51(D1):D1373-d80.
40. Berman H M, Westbrook J, Feng Z, Gilliland G, Bhat T N, Weissig H, et al. The Protein Data Bank. Nucleic Acids Res. 2000; 28(1):235-42.
41. Gordon J C, Myers J B, Folta T, Shoja V, Heath L S, Onufriev A. H++: a server for estimating p Ka s and adding missing hydrogens to macromolecules. Nucleic Acids Research. 2005; 33(suppl_2):W368-W71.
42. MetaPocket: A Meta Approach to Improve Protein Ligand Binding Site Prediction. OMICS: A Journal of Integrative Biology. 2009; 13(4):325-30.
43. Stroganov O V, Novikov F N, Stroylov V S, Kulkov V, Chilov G G. Lead finder: an approach to improve accuracy of protein-ligand docking, binding energy estimation, and virtual screening. J Chem Inf Model. 2008; 48(12):2371-85.
44. Maier J A, Martinez C, Kasavajhala K, Wickstrom L, Hauser K E, Simmerling C. ff14SB: Improving the Accuracy of Protein Side Chain and Backbone Parameters from ff99SB. Journal of Chemical Theory and Computation. 2015; 11(8):3696-713.
45. Wang J, Wolf R M, Caldwell J W, Kollman P A, Case D A. Development and testing of a general amber force field. J Comput Chem. 2004; 25(9):1157-74.
46. Case D A, Cheatham T E, 3rd, Darden T, Gohlke H, Luo R, Merz K M, Jr., et al. The Amber biomolecular simulation programs. J Comput Chem. 2005; 26(16):1668-88.
47. Yoshimura N, Kudoh S, Kimura T, Mitsuoka S, Matsuura K, Hirata K, et al. EKB-569, a new irreversible epidermal growth factor receptor tyrosine kinase inhibitor, with clinical activity in patients with non-small cell lung cancer with acquired resistance to gefitinib. Lung Cancer. 2006; 51(3):363-8.
48. Günther S, Reinke P Y A, Fernández-García Y, Lieske J, Lane T J, Ginn H M, et al. X-ray screening identifies active site and allosteric inhibitors of SARS-CoV-2 main protease. Science. 2021; 372(6542):642-6.
49. Ertl P, Rohde B, Selzer P. Fast calculation of molecular polar surface area as a sum of fragment-based contributions and its application to the prediction of drug transport properties. J Med Chem. 2000; 43(20):3714-7.
50. Lipinski C A, Lombardo F, Dominy B W, Feeney P J. Experimental and computational approaches to estimate solubility and permeability in drug discovery and development settings. Adv Drug Deliv Rev. 2001; 46(1-3):3-26.
51. Chan J F, Kok K H, Zhu Z, Chu H, To K K, Yuan S, et al. Genomic characterization of the 2019 novel human-pathogenic coronavirus isolated from a patient with atypical pneumonia after visiting Wuhan. Emerg Microbes Infect. 2020; 9(1):221-36.
52. Hoffman R L, Kania R S, Brothers M A, Davies J F, Ferre R A, Gajiwala K S, et al. Discovery of Ketone-Based Covalent Inhibitors of Coronavirus 3CL Proteases for the Potential Therapeutic Treatment of COVID-19. J Med Chem. 2020; 63(21):12725-47.
53. Gorbalenya A E, Donchenko A P, Blinov V M, Koonin E V. Cysteine proteases of positive strand RNA viruses and chymotrypsin-like serine proteases. A distinct protein superfamily with a common structural fold. FEBS Lett. 1989; 243(2):103-14.
54. Chou C Y, Chang H C, Hsu W C, Lin T Z, Lin C H, Chang G G. Quaternary structure of the severe acute respiratory syndrome (SARS) coronavirus main protease. Biochemistry. 2004; 43(47):14958-70.
55. Hsu W C, Chang H C, Chou C Y, Tsai P J, Lin P I, Chang G G. Critical assessment of important regions in the subunit association and catalytic action of the severe acute respiratory syndrome coronavirus main protease. J Biol Chem. 2005; 280(24):22741-8.
56. Tan J, Verschueren K H, Anand K, Shen J, Yang M, Xu Y, et al. pH-dependent conformational flexibility of the SARS-CoV main proteinase (M(pro)) dimer: molecular dynamics simulations and multiple X-ray structure analyses. J Mol Biol. 2005; 354(1):25-40.
57. Shi J, Sivaraman J, Song J. Mechanism for controlling the dimer-monomer switch and coupling dimerization to catalysis of the severe acute respiratory syndrome coronavirus 3C-like protease. J Virol. 2008; 82(9):4620-9.
58. Nilsson I, Saaf A, Whitley P, Gafvelin G, Waller C, von Heijne G. Proline-induced disruption of a transmembrane alpha-helix in its natural environment. J Mol Biol. 1998; 284(4):1165-75.
59. Noske G D, Song Y, Fernandes R S, Chalk R, Elmassoudi H, Koekemoer L, et al. An in-solution snapshot of SARS-COV-2 main protease maturation process and inhibition. Nat Commun. 2023; 14(1):1545.
60. Ferreira G M, Kronenberger T, Tonduru A K, Hirata R D C, Hirata M H, Poso A. SARS-COV-2 M(pro) conformational changes induced by covalently bound ligands. J Biomol Struct Dyn. 2022; 40(22):12347-57.
61. Li C, Qi Y, Teng X, Yang Z, Wei P, Zhang C, et al. Maturation mechanism of severe acute respiratory syndrome (SARS) coronavirus 3C-like proteinase. J Biol Chem. 2010; 285(36):28134-40.
62. Cheng S C, Chang G G, Chou C Y. Mutation of Glu-166 blocks the substrate-induced dimerization of SARS coronavirus main protease. Biophys J. 2010; 98(7):1327-36.

1. A composition comprising a compound having formula (I): 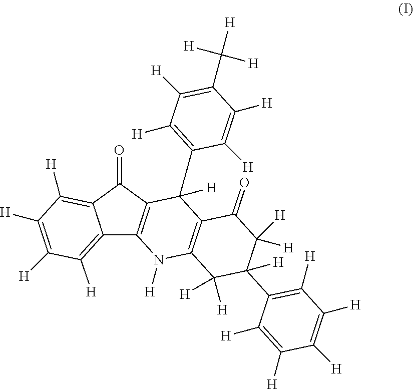 [SMILES: Cc1ccc(C2C3=C(CC(c4ccccc4)CC3=O)NC3=C2C(=O)c2ccccc23)cc1]
(I) or a derivative thereof;
in an effective amount to inhibit a human coronavirus when administered in vivo.

2. The composition of claim 1, wherein the coronavirus is selected from the group consisting of SARS-CoV-2, MERS-CoV, SARS-CoV and HCoV-299E.

3. The composition of claim 1, wherein the composition is a unit dosage form.

4. The composition of claim 3, wherein the unit dosage form is a tablet or capsule.

5. A method of treating coronavirus infection and/or preventing growth of coronavirus in a subject, the method comprising administering an antiviral composition comprising an effective amount of a compound of Formula (I): 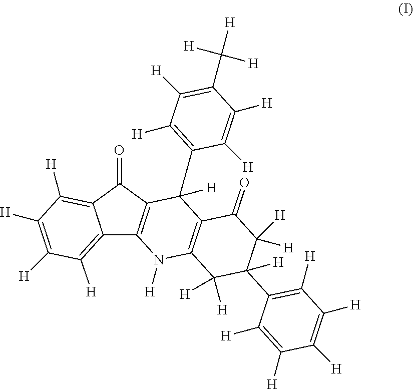 [SMILES: Cc1ccc(C2C3=C(CC(c4ccccc4)CC3=O)NC3=C2C(=O)c2ccccc23)cc1]
(I), or a derivative thereof,
to the subject.

6. The method of claim 5, wherein the subject is infected with SARS-CoV-2.

7. The method of claim 5, wherein the subject is infected with MERS-CoV.

8. The method of claim 5, wherein the subject is infected with HCoV-299E.

9. The method of claim 5, further comprising administering nirmatrelvir to the subject.

10. The method of claim 9, wherein the antiviral composition further comprises nirmatrelvir.

11. The method of claim 5, wherein the wherein the composition is administered parenterally or orally.

12. The method of claim 5, wherein the wherein the composition is administered intranasally or by pulmonary administration.
